(19) United States
(12) Patent Application Publication
(10) Pub. No.: US 2006/0205032 A1
(43) Pub. Date: Sep. 14, 2006

(54) hSLIM modulates immune responses

(75) Inventors: Mary Faris, Los Angeles, CA (US); Sandra Aeyoung Lee, Sherman Oaks, CA (US); Zhiyong Qiu, Los Angeles, CA (US); David Brandt, Moorpark, CA (US)

Correspondence Address: BANNER & WITCOFF, 1001 G STREET N W, SUITE 1100, WASHINGTON, DC, 20001, US

(73) Assignee: MannKind Corporation, Valencia, CA (US)

(21) Appl. No.: 11/183,324

(22) Filed: Jul. 18, 2005

Related U.S. Application Data
(60) Provisional application No. 60/668,984, filed on Apr. 7, 2005.
(60) Provisional application No. 60/659,873, filed on Mar. 10, 2005.

(51) Int. Cl. C12P 21/06; C07H 21/04; C07K 14/705

(52) U.S. Cl. 435/69.1; 435/320.1; 435/325; 530/350; 536/23.5

(57) ABSTRACT

Human SLIM polypeptides for use in modulating STAT-dependent gene transcription.

This application claims the benefit of and incorporates by reference provisional applications Ser. No. 60/659,873 filed Mar. 10, 2005 and Ser. No. 60/668,984 filed Apr. 7, 2005.

### FIELD OF THE INVENTION

The invention relates to tools and methods for modulating immune responses.

### BACKGROUND OF THE INVENTION

STAT proteins (signal transducers and activators of transcription) mediate both signal transduction and act as transcription factors (Ihle et al., Trends Biochem Sci 19, 222-27, 1994). Their association with specific phosphotyrosine peptides on the cytoplasmic domain of cytokine receptors activates these factors. Upon association with these peptides the STATs become phosphorylated by Jak tyrosine kinases. The activated STATs act as transcription factors and bind to DNA as dimers. The action of several cytokines including gamma interferon, interleukin-1, and interleukin-6, is mediated at least in part by STAT proteins. It would, therefore, be useful for therapeutic purposes to have methods of regulating STAT signal transduction and STAT-dependent transcription and methods of screening for agents which regulate these functions.

### BRIEF DESCRIPTION OF THE FIGURES

FIG. 1. A nucleic acid sequence (SEQ ID NO:1) encoding wild-type hSLIM (SEQ ID NO:2). The nucleotide sequence encoding the PDZ domain is bolded and underlined. The nucleotide sequence encoding the LIM domain is italicized and underlined.

FIG. 2A. The amino acid sequence (SEQ ID NO:2) of wild-type hSLIM (isoform 1). The PDZ domain is bolded and underlined. The LIM domain is italicized and underlined. FIG. 2B, amino acid sequence of an hSLIM isoform (SEQ ID NO:3). The PDZ domain is bolded and underlined. The LIM domain is italicized and underlined.

FIG. 3. Linear Schematic of human SLIM domains. Protein-binding PDZ domain, amino acids 5-82 of SEQ ID NO:2; zinc-binding LIM domain, amino acids 284-337 of SEQ ID NO:2. Theoretical pI/Mw, 8.76/37.5 kDa.

FIG. 4. Alignments of wild-type hSLIM (SEQ ID NO:2) (Query) with mystique isoforms (identified by GenBank Accession numbers; Sbjct). FIG. 4A, alignment of wild-type hSLIM (SEQ ID NO:2) with mouse AAH24556 or AAL65265 (SEQ ID NO:8). 529 bits (1363), Expect=e-149, Identities=277/354 (78%), Positives=299/354 (84%), Gaps=7/354 (1%). FIG. 4B, alignment of wild-type hSLIM (SEQ ID NO:2) with human NP067643 (SEQ ID NO:2). Identities=277/354 (78%), Positives=299/354 (84%), Gaps=7/354 (1%). FIG. 4C, alignment of wild-type hSLIM (SEQ ID NO:2) with Pan troglodytes XP519648 (SEQ ID NO:9). Score=423 bits (1088), Expect=e-117, Identities=224/235 (95%), Positives=224/235 (95%), Gaps=1/235 (0%). FIG. 4D, alignment of wild-type hSLIM (SEQ ID NO:2) with human NP932159 (SEQ ID NO:5). Score=497 bits (1279), Expect=e-139, Identities=255/255 (100%), Positives =255/255 (100%). FIG. 4E, alignment of wild-type hSLIM (SEQ ID NO:2) with human NP789847 (SEQ ID NO:6). Score=650 bits (1677), Expect=0.0, Identities=327/327 (100%), Positives=327/327 (100%). FIG. 4F, alignment of wild-type hSLIM (SEQ ID NO:2) with human AAL65265 (SEQ ID NO:7). Score=464 bits (1193), Expect=e-129, Identities=237/242 (97%), Positives=238/242 (97%). FIG. 4G, alignment of wild-type hSLIM (SEQ ID NO:2) with human AAG16633 (SEQ ID NO:3). Score=582 bits (1499), Expect=e-164, Identities=304/352 (86%), Positives=307/352 (86%), Gaps=27/352 (7%).

FIG. 5. Agarose gel showing RT-PCT hSLIM expression products in select human tissues. Primer locations are shown at the right. Isoform 1 (wild-type hSLIM) is preferentially expressed in lung (top band). Isoform 3 is preferentially expressed in CD4+ T cells and in spleen (lower band).

FIG. 6. Agarose gel showing RT-PCT hSLIM expression products in CD4+ and E6.1 cells.

FIG. 7. Alignment of portions of hSLIM isoforms. Isoform 1, full-length, wild-type hSLIM (SEQ ID NO:2), which is differentially expressed in lung; Isoform 3, partial deletion that maintains PDZ and LIM domains (SEQ ID NO:4), which is expressed in CD4+ cells; Isoform 2 (fragment shown, SEQ ID NO:10), which is truncated after the PDZ domain.

FIG. 8. Anti-hSLIM Western blot showing expression of human SLIM protein in various expression systems. FIG. 8A, bacterial expression system; FIG. 8B, mammalian expression system; FIG. 8C, baculoviral expression system.

FIG. 9. Effect of RNAi on endogenous hSLIM Expression in Jurkat E6.1 Cells. The human Jurkat T cell line was transfected with control or SLIM-specific (SL1 and SL2) RNAi. Transfection efficiency was evaluated by FACS. Cell toxicity and protein knock down were evaluated by cell titer and western blotting, respectively. Transfection efficiency was 74% in E6.1 cells, with no apparent toxicity or off target effect. Western blot analysis shows loss of hSLIM protein in cells transfected with 3-6 μg of SLIM RNAi. Expression of unrelated genes remained constant in control and transfected cells, showing lack of off target effect.

FIG. 10. Effect of RNAi on hSLIM Protein Expression in CD4+T Cells. Human CD4+T cells were purified from buffy coat and transfected with control (Scr and FITC) or SLIM-specific(SL2) RNAi. Transfection efficiency was evaluated by FACS. SLIM knock down was evaluated by RT-PCR, and off target effect by western blotting, respectively. Transfection efficiency was 79% in cells transfected with 6μg RNAi. PCR analysis shows loss of hSLIM mRNA in cells transfected with 6 μg of SLIM RNAi. Expression of unrelated genes remained constant in control and transfected cells, showing lack of off target effect.

FIG. 11. Effect of SLIM Knock Down. FIG. 11A, effect of SLIM knock down on IFNγ Production in CD4+T cells. Untransfected CD4+T cells or cell transfected with SLIM or control RNAi were treated with anti-CD3+anti-CD28 for 48 hr, washed and rested for 5 days. Cells were either left unstimulated or treated with anti-CD3+anti-CD28 for 24 h prior to FACS analysis. Partial Knock down of SLIM expression enhances IFNγproduction. FIG. 11B, Effect of SLIM Knock Down on IFNγ Production in CD4+ T cells. Untransfected CD4+ T cells or cell transfected with SLIM or control RNAi were treated with anti-CD3+anti-CD28 for 48 hr, washed and rested for 5 days. Cells were either left unstimulated or treated with anti-CD3+anti-CD28 for the indicated amount of time prior to ELISA analysis. Partial Knock down of SLIM expression enhances IFNγ production.

FIG. 12. E3 Ligase Activity of hSLIM. Ubiquitination of recombinant SLIM Protein. GST-SLIM protein was incubated in the presence of E1, E2 and ubiquitin at 30° C. Proteins were separated on NuPage gels and blotted with Avidin-HRP. The blot was overlayed with anti-SLIM pAb to show SLIM loading. hSLIM demonstrates E3 ligase activity by inducing its own ubiquitination. Ubiquitin transfer occurs in a dose dependent manner.

FIG. 13. Effect of Zinc and EDTA on Ubiquitin Transfer. GST-SLIM protein was treated with Zinc or EDTA for 2 hr, washed and incubated in the presence of E1, E2 and ubiquitin at 30° C. Proteins were separated on NuPage gels and blotted with Avidin-HRP. The blot was overlayed with anti-SLIM pAb. The E3 ligase activity of hSLIM was improved upon treatment of the protein with zinc. In contrast, EDTA inhibited the enzyme activity of hSLIM.

FIG. 14. Generation of LIM-domain point mutants. Alignment of hSLIM LIM domain (SEQ ID NO:20) with Ring motifs from other proteins (SEQ ID NOS:21-23) revealed the presence of conserved cysteines at positions corresponding to C1 and C2 of the consensus site. These 2 cyteines, C286 and C289, were mutated to serines, thereby generating a LIM-domain point mutant of hSLIM.

FIG. 15. Ubiquitin Transfer Activities of WT- and Mu-SLIM, aka Mu-SLIM C1C2. We compared the E3 ligase activity of wild-type (WT) and point mutant (Mu) GST-SLIM protein. The reaction was performed as previously described. Proteins were separated on NuPage gels and blotted with Avidin-HRP. Compared to WT-SLIM, the E3 ligase activity of hSLIM was reduced when C1 and C2 positions were mutated to serine. These results indicate the relevance of an intact LIM domain for activity, and suggest that disrupting the 3D structure of hSLIM prevents enzyme activity.

FIG. 16. Ubiquitination of STAT1 by rhSLIM. We compared the ability of WT- and Mu-SLIM C1C2 protein to ubiquitinate a physiologic substrate, STAT1. Recombinant hSLIM was incubated in the presence of various doses of STAT1, and the reaction was performed as previously described. Proteins were separated on NuPage gels and blotted with Avidin-HRP. WT-SLIM mediates the ubiquitination of STAT1 and demonstrates strong E3 ligase activity towards both STAT1 and hSLIM. The ubiquitin transfer ability of Mu-SLIM C1C2 was reduced relative to WT-SLIM.

FIG. 17. Regulation of STAT4 Transcriptional Activity by WT- and Mu-SLIM C1C2. FIG. 17A: 293T cells were transiently transfected with STAT4 or STAT4+WT- or Mu-SLIM, along with reporter constructs encoding the GAS-response element. Cells were either left untreated, or stimulated with IFNα, and analyzed for GAS-Luc activity. Treatment with IFNα enhances STAT4-mediated GAS-luc activity, which is inhibited by WT-SLIM in a dose dependent manner. In contrast, Mu-SLIM has little effect on GAS-Luc activity. FIG. 17B: STAT4 Phosphorylation in 293T Cells. 293T cells were transiently transfected with STAT4 or with STAT4+WT- or Mu-SLIM, and were either left untreated, or stimulated with IFNα. Cell lysates were immunoprecipitated with anti-STAT4 or control Ab and analyzed for STAT4 phosphorylation. Treatment with IFNα enhances STAT4 phosphosylation in 293T cells.

FIG. 18. Regulation of STAT1 Transcriptional Activity by WT- and Mu-SLIM C1C2. 293T cells were transiently transfected with STAT1 or STAT1+WT- or Mu-SLIM, along with reporter constructs encoding the GAS-response element. Cells were either left untreated, or stimulated with IFNγ, and analyzed for GAS-Luc activity. Treatment with IFNγ enhances STAT1-mediated GAS-luc activity, which is inhibited by WT-SLIM in a dose dependent manner. In contrast, Mu-SLIM has little effect on GAS-Luc activity.

FIG. 19. Association of SLIM and STAT1 in 293T Cells. 293T cells were transiently tranfected STAT1 alone, or with HA-SLIM and STAT1. Cells were either left untreated or treated with IFNγ for 15 min. Cells were lysed, immunoprecipitated with anti-STAT1 Ab and blotted for the presence of HA-SLIM. STAT1 and SLIM co-precipitate when expressed simultaneously in 293T cells.

FIG. 20. Association of SLIM and STAT4 in 293T cells. 293T cells were transiently tranfected STAT4 alone or with HA-SLIM and STAT4. Cells were either left untreated or treated with IFNα for 20 min. Cells were lysed, immunoprecipitated with anti-STAT4 Ab and blotted for the presence of HA-SLIM. Results: STAT4 and SLIM co-precipitate when expressed simultaneously in 293T cells.

FIG. 21. Generation of hSLIM-specific antibodies. In order to generate antibodies specific to human SLIM and minimize cross reactivity to other PDZ- and LIM- domain proteins, we generated a vector expressing human SLIM lacking the PDZ and LIM domains, i.e. DPDZ-DLIM-SLIM. The His-Tagged protein was introduced into rabbits for pAb production.

FIG. 22. Multiple sequence alignments of mystique nucleic acid sequences. FIG. 22A, CLUSTAL W (1.74) alignment of gi|47940542|gb|BC071774.1 (SEQ ID NO:27) and gi|40288188|ref|NM_{—}021630.4| (SEQ ID NO:28). FIG. 22B, alignment of gi|40288188|ref|NM_{—}021630.41| (SEQ ID NO:28), gi|40288187|ref|NM_{—}176871.2| (SEQ ID NO:29), and gi|40288186|ref|NM_{—}198042.2 (SEQ ID NO:30). FIG. 22C, alignment of gi|40288186|ref|MN_{—}198042.2 (SEQ ID NO:30), gi|18204288|gb|BC021556.1 (SEQ ID NO:31), gi|2751684|dbj|AK092968.1 (SEQ ID NO:32), gi|16552238|dbj|AK056748.1| (SEQ ID NO:33), gi|33151167|gb|AY070438.1| (SEQ ID NO:34), and gi|10445214|gb|AY007729.1 (SEQ ID NO:35); FIG. 22D, alignment of gi|47940542|gb|BC071774.1| (SEQ ID NO:27), gi|40288186|ref|NM_{—}198042.2 (SEQ ID NO:30), gi|18204288|gb|BC021556.1 (SEQ ID NO:37), gi|16552238|dbj|AK056748.1| (SEQ ID NO:38), gi|33151167|gb|AY070438.1| (SEQ ID NO:40), gi|10445214|gb|AY007729.1 (SEQ ID NO:39); FIG. 2E, alignment of wt-SLIM (SEQ ID NO:1), gi|221224221|ref|NM_{—}145978.1| (SEQ ID NO:36), gi|19354024|gb|BC024556.1| (SEQ ID NO:37), gi|55630341|ref|XM_{—}519648.1| (SEQ ID NO:38), and gi|55167726|gb|BV210713.1| (SEQ ID NO:39).

FIG. 23. Clustal alignment of mystique proteins. g|21361888|ref|NP_{—}067643.2| (SEQ ID NO:41), gi|383276121|ref|NP_{—}932159.1| (SEQ ID NO:42), gi|28866957|ref|NP_{—}789847.1| (SEQ ID NO:43), gi|33151168|gb|AAL65265.1| (SEQ ID NO:44), gi|10445215|gb|AAG16633.1| (SEQ ID NO:5), WT-SLIM (SEQ ID NO:2), hSLIM-D-52deletion (SEQ ID NO:4), gi|22122423|ref|NP_{—}666090.1| (SEQ ID NO:46), gi|19354025|gb|AAH24556.1| (SEQ ID NO:47), gi|55630342|ref|XP_{—}519648.1|, and (SEQ ID NO:48).

FIG. 24. Western blots showing E3 ligase activity of SLIM isoform WT-Δ52-SLIM.

FIG. 25. Western blots showing ubiquitination of STAT-1 by WT-hSLIM and WT-SLIM-Δ52.

FIG. 26. Western blots showing identification of human E2 enzes that facilitate WT-SLIM-Δ52 ubiquitination.

FIG. 27. Comparison of ring domain sequences of wild-type hSLIM and various hSLIM mutants. “SLIM Ring Domain” and WT-SLIM, SEQ ID NO:50; Mu-SLIMC1C2, SEQ ID NO:51; Mu-SLIM C5C6, SEQ ID NO:52; and MuSLIM C1C2C5C6, SEQ ID NO:53.

FIG. 28. Sypro ruby stained gel (FIG. 28A) and the anti-SLIM western blot (FIG. 28B) demonstrate expression and purification of recombinant hSLIM protein.

FIG. 29. Avidin-HRP blot (FIG. 29A) and anti-hSLIM blot (FIG. 29B).

FIG. 30. Avidin-HRP blot (FIG. 30A) and anti-SLIM blot (FIG. 30B).

FIG. 31. Bar graph showing the effect of wild-type and mutant SLIM on STAT1 transcriptional activity.

FIG. 32. Anti-SLIM blot.

FIG. 33. Anti-UbcH5 blot (FIG. 33A) and anti-UbcH6 blot (FIG. 33B).

### SUMMARY OF THE INVENTION

The invention provides isolated and purified polypeptides. In some embodiments the polypeptides comprise the amino acid sequence shown in SEQ ID NO:4. In other embodiments the polypeptides comprise the amino acid sequence shown in SEQ ID NO:49. In still other embodiments an isolated and purified polypeptide consists of the amino acid sequence shown in SEQ ID NO:19. The invention also provides fusion proteins comprising any of these polypeptides and a second polypeptide which is not wild-type hSLIM as shown in SEQ ID NO:2.

In other embodiments the invention provides isolated and purified nucleic acid molecules which comprise a coding sequence for any of these polypeptides or the complement of the coding sequence; the coding sequence does not encode SEQ ID NOS:2, 3, 5-9, or 41-45. The nucleic acid molecules can be, for example, double-stranded molecules, cDNA, or RNA. The invention also provides vectors comprising the nucleic acid molecules. The vectors can be, e.g., bacterial vectors, mammalian vector, baculovirus vectors. The nucleic acid molecules also can be present in an expression construct comprising a coding sequence for a polypeptide of the invention and a promoter which is located upstream from the coding sequence and which controls expression of the coding sequence.

The invention provides host cells comprising the expression constructs. The host cells can be, for example, mammalian, human, bacterial, or insect cells. The invention provides methods of making polypeptides of the invention which comprise culturing a host cell in a culture medium under conditions whereby the host cell expresses the polypeptide and recovering the polypeptide from the culture medium or a host cell lysate. Optionally, the polypeptide can be purified and/or refolded in the presence of zinc or in the absence of EDTA, DTPA, TPEN, and EGTA.

The invention provides a method of producing enzymatically active wild-type hSLIM comprising the amino acid sequence shown in SEQ ID NO:2. The method comprises purifying the wild-type hSLIM and/or refolding the wild-type hSLIM in the presence of zinc. Purification and/or refolding can be conducted in the absence of EDTA, DTPA, TPEN, and EGTA.

Other embodiments of the invention are antibodies which specifically bind to a portion of an hSLIM polypeptide which is not a PDZ or a LIM domain. The antibodies can be polyclonal, monoclonal, chimeric, humanized, of human antibodies. They can be Fab, F(ab′)₂, or Fv fragments, single-chain antibodies, or intracellular antibodies. Optionally the antibodies can comprise a detectable label.

The invention also provides methods for identifying compounds which interfere with binding of an hSLIM polypeptide to a STAT protein. In one embodiment a first polypeptide, a second polypeptide, and a test compound are contacted. Either (1) the first polypeptide comprises an hSLIM polypeptide and the second polypeptide comprises a STAT protein; or (2) the first polypeptide comprises the STAT protein and the second polypeptide comprises the hSLIM polypeptide. The quantity of the first polypeptide which is bound to, is displaced from, or is prevented from binding to, the second polypeptide is determined. The test compound is identified as an agent which interferes with the binding of the hSLIM polypeptide to the STAT protein if the test compound (1) diminishes the quantity of the first polypeptide bound to the second polypeptide; (2) displaces first polypeptide bound to the second polypeptide; or (3) prevents first polypeptide from binding to the second polypeptide. An antibody can be used to determine the quantity of the first polypeptide which is (1) bound to, (2) displaced from, or (3) prevented from binding to the second polypeptide. In some embodiments the antibody specifically binds to the hSLIM polypeptide. In other embodiments the antibody specifically binds to the STAT protein. Either the first or the second polypeptide can be fixed to a solid support. Optionally, one of the polypeptides comprises a detectable label. Also, optionally, one or both of the first and second polypeptides can be a fusion protein. In some embodiments the first polypeptide comprises the hSLIM polypeptide and the second polypeptide comprises STAT1 or STAT4. In other embodiments the first polypeptide comprises STAT1 or STAT4 and the second polypeptide comprises the hSLIM polypeptide.

The invention also provides methods for identifying compounds which interfere with STAT1- or STAT4-mediated transcription. A test compound, a first polypeptide, a second polypeptide, and a reporter construct comprising a STAT1- or STAT4-binding sequence upstream from a reporter gene are contacted to form a transcription mixture. Either (1) the first polypeptide comprises an hSLIM polypeptide and the second polypeptide comprises STAT1 or STAT4; or (2) the first polypeptide comprises STAT1 or STAT4 and the second polypeptide comprises the hSLIM polypeptide. The transcription mixture is contacted with IFNα if the transcription mixture comprises STAT4 or the transcription mixture is contacted with IFNγ if the transcription mixture comprises STAT1. Expression of the reporter gene is assayed. The test compound is identified as an agent which interferes with STAT1- or STAT4-mediated transcription if expression of the reporter gene in the presence of the test compound is less than expression of the reporter gene in the absence of the test compound. The STAT1- or STAT4-binding sequence can be a GAS sequence. The step of contacting can be in a cell or in a cell-free system. The methods also can comprise assaying the ability of the test compound to modulate IFNγ production, STAT1- or STAT4-mediated transcription, STAT1 or STAT4 phosphorylation, Th1 or Th2 cell differentiation, or T-bet-activity.

Other embodiments of the invention provide methods for identifying compounds which interfere with binding of hSLIM to STAT1 or STAT4. A test compound and a cell which comprises three recombinant DNA constructs are contacted. A first construct encodes a first polypeptide fused to a sequence-specific DNA-binding domain; a second construct encodes a second polypeptide fused to a transcriptional activation domain; and a third construct comprises a reporter gene downstream from a DNA element which is recognized by the sequence-specific DNA-binding domain. Either the first polypeptide comprises an hSLIM polypeptide and the second polypeptide comprises STAT1 or STAT4; or the first polypeptide comprises STAT1 or STAT4 and the second polypeptide comprises the hSLIM1 polypeptide. The cell is contacted with the test compound and expression of the reporter gene in the presence of the test compound is determined. The test compound as an agent which interferes with the binding of hSLIM to STAT1 or STAT4 if the expression of the reporter gene in the presence of the test compound is less than expression of the reporter gene in the absence of the test compound. The methods also can comprise assaying the ability of the test compound to modulate IFNγ production, STAT1- or STAT4-mediated transcription, STAT1 or STAT4 phosphorylation, Th1 or Th2 cell differentiation, or T-bet-activity.

Another embodiment of the invention is a cell comprising three recombinant DNA constructs. A first construct encodes a first polypeptide comprising a sequence-specific DNA-binding domain; a second construct encodes a second polypeptide comprising a transcriptional activation domain; and a third construct comprises a reporter gene downstream from a DNA element which is recognized by the sequence-specific DNA-binding domain, wherein either the first polypeptide comprises an hSLIM polypeptide and the second polypeptide comprises a STAT protein; or the first polypeptide comprises the STAT protein and the second polypeptide comprises the hSLIM polypeptide. The STAT protein can be, for example, STAT1 or STAT4.

The invention also provides methods of identifying agonists or antagonists of E3 ligase activity of hSLIM. An hSLIM polypeptide is contacted with a test compound, and the E3 ligase activity of the hSLIM polypeptide is assayed. The test compound is identified as an agonist of hSLIM E3 ligase activity if the test compound increases E3 ligase activity of the hSLIM polypeptide relative to E3 ligase activity of the hSLIM polypeptide in the absence of the test compound. The test compound is identified as an antagonist of hSLIM E3 ligase activity if the test compound decreases E3 ligase activity of the hSLIM polypeptide relative to E3 ligase activity of the hSLIM polypeptide in the absence of the test compound. Ligase activity can be assayed, for example, by detecting ubiquitination of the hSLIM polypeptide or by detecting ubiquitination of a STAT protein. The STAT protein can be, e.g., STAT1 or STAT4. In some embodiments ubiquitination is detected using fluorescence resonance energy transfer. In other embodiments ubiquitination is detected using a DELFIA assay. In other embodiments an alpha screen is used. The methods also can comprise assaying the ability of the test compound to modulate IFNγ production, STAT1- or STAT4-mediated transcription, STAT1 or STAT4 phosphorylation, Th1 or Th2 cell differentiation, or T-bet-activity.

In screening methods of the invention the hSLIM polypeptide can be, e.g., a wild-type hSLIM, WT-A52-hSLIM (SEQ ID NO:4).

The invention provides compositions comprising an active agent, an immunomodulatory agent, and a physiologically acceptable vehicle. The active agent can be, e.g., (1) an hSLIM polypeptide; (2) a nucleic acid molecule encoding the hSLIM polypeptide; (3) an siRNA molecule which silences transcription of an hSLIM gene; (4) an antisense oligonucleotide which prevents transcription of an hSLIM gene; (5) an antibody which specifically binds to an hSLIM polypeptide. The immunomodulatory agent can be, for example, (1) a vaccine; (2) a dendritic cell; or (3) a monoclonal antibody. Other compositions of the invention comprise an siRNA molecule selected from the group consisting of SEQ ID NOS:15 and 16 and a physiologically acceptable vehicle. The siRNA molecule can be in a vector, which can be, for example, a lentivirus vector, a retrovirus vector, or an adenovirus vector. The physiologically acceptable vehicle can be non-pyrogenic. The hSLIM polypeptide can be, e.g., a wild-type hSLIM, WT-Δ52-hSLIM (SEQ ID NO:4).

Other embodiments of the invention provide methods of altering (e.g., decreasing or increasing) IFNγ production by a T cell. In some embodiments the T cell is contacted with an hSLIM polypeptide or a nucleic acid molecule encoding the hSLIM polypeptide; these methods decrease IFNγ production by the T cell. In other embodiments the T cell is contacted with a reagent selected from the group consisting of (a) the antibody of claim 23; (b) an siRNA molecule selected from the group consisting of SEQ ID NOS:9 and 10; and (c) an antisense oligonucleotide which hybridizes to a portion of SEQ ID NO: 1 or SEQ ID NO:27; in these methods IFNγ production by the T cell is increased. In any of these embodiments the T cell can be a Th1 or Th2 cell. The methods can be carried out in vitro or in vivo.

### DETAILED DESCRIPTION OF THE INVENTION

hSLIM Polypeptides

The invention provides, inter alia, hSLIM polypeptides which are useful for modulating STAT-dependent gene expression. An “hSLIM polypeptide” according to the invention includes wild-type hSLIM as shown in SEQ ID NO:2 and hSLIM isoforms as shown in SEQ ID NOS:3-7, as well as polypeptides having amino acid sequences which are between 78 and 99% identical to SEQ ID NO:2 (e.g., 78, 79, 80, 85, 90, 95, 96, 97, 98, or 99% identical to SEQ ID NO:2). An hSLIM polypeptide of the invention preferably has one or more of the following functions: it binds to a STAT protein (particularly STAT1 and STAT4); it transfers a ubiquitin molecule to itself by means of an E3 ligase activity; it transfers a ubiquitin molecule to a STAT protein (particularly STAT1 or STAT4) by means of an E3 ligase activity; it inhibits STAT-mediated transcription (particularly IFNγ-enhanced STAT1- and IFNα-enhanced STAT4-mediated transcription); it affects various functions such as STAT phosphorylation, T-bet activity, Th1 or Th2 cell differentiation, and IFNγ production.

In some embodiments, an hSLIM polypeptide differs from wild-type hSLIM as shown in SEQ ID NO:2 by between one and 50 conservative amino acid substitutions (e.g., 1, 2, 3, 4, 5, 6, 7, 8, 9, 10 or between 1 and 25, 1 and 15, 1 and 10, or 1 and 5 substitutions). Examples of conservative substitutions include, but are not limited to, Gly⇄Ala, Val⇄Ile⇄Leu, Asp⇄Glu, Lys⇄Arg, Asn⇄Gln, and Phe⇄Trp⇄Tyr. Conservative amino acid substitutions typically fall in the range of about 1 to 5 amino acids (i.e., 1, 2, 3, 4, or 5 amino acids). Additional amino acids can be added at any position in the molecule, particularly at the amino- or carboxy terminus. Amino acid additions can be 1, 2, 5, 10, 25, 100, or more additional amino acids. An hSLIM polypeptide preferably comprises TCEKCST (SEQ ID NO:24) and/or RHPGCYTCA (SEQ ID NO:25). More preferably an hSLIM polypeptide comprises amino acids 284-337 of SEQ ID NO:2.

One particular hSLIM polypeptide of interest (WT-Δ52-hSLIM or A52-(h)SLIM) is shown in SEQ ID NO:4; this polypeptide has a 52 amino acid deletion relative to wild-type hSLIM as shown in SEQ ID NO:2. See Example 2.

hSLIM polypeptides also include those with point mutations (see Example 3 and SEQ ID NO:26).

Fusion Proteins Comprising hSLIM Polypeptides

An hSLIM polypeptide can be present in a fusion protein. Such fusion proteins comprise two polypeptide segments fused together by means of a peptide bond. The first polypeptide segment is an hSLIM polypeptide as defined above. The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the hSLIM polypeptide-encoding sequence and the heterologous protein sequence, so that the hSLIM polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises a coding sequence for an hSLIM polypeptide in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

hSLIM Nucleic Acid Molecules

hSLIM nucleic acid molecules can be single- or double-stranded and can be either RNA or DNA molecules. hSLIM nucleic acid molecules comprise a coding sequence or the complement of a coding sequence for an hSLIM polypeptide. A coding sequence for wild-type hSLIM (SEQ ID NO:2) is shown in SEQ ID NO: 1. Other hSLIM coding sequences are shown in SEQ ID NOS:27-35 and 40. Because of the degeneracy of the genetic code, however, any nucleotide sequence which encodes an hSLIM polypeptide can be used.

Preparation of hSLIM Nucleic Acid Molecules

hSLIM nucleic acid molecules can be isolated free of other cellular components such as membrane components, proteins, and lipids. hSLIM nucleic acid molecules can be made by a cell and isolated using standard nucleic acid purification techniques, or synthesized using an amplification technique, such as the polymerase chain reaction (PCR), or by using an automatic synthesizer. Methods for isolating nucleic acid molecules are routine and are known in the art. Any such technique for obtaining a nucleic acid molecule can be used to obtain isolated hSLIM nucleic acid molecules. Isolated hSLIM nucleic acid molecules are in preparations which are free or at least 70, 80, or 90% free of other molecules.

hSLIM cDNA molecules can be made with standard molecular biology techniques, using hSLIM mRNA as a template. hSLIM cDNA molecules can thereafter be replicated using molecular biology techniques known in the art and disclosed in manuals such as Sambrook et al. (1989). An amplification technique, such as PCR, can be used to obtain additional copies of nucleic acid molecules of the invention, using either human genomic DNA or cDNA as a template.

Alternatively, synthetic chemistry techniques can be used to synthesize hSLIM nucleic acid molecules. Sequences encoding an hSLIM polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980), which are incorporated by reference in their entireties herein. The degeneracy of the genetic code allows alternate nucleotide sequences to be synthesized which will encode an hSLIM polypeptide.

Preparation of hSLIM Polypeptides

hSLIM polypeptides can be obtained, for example, by purification from human cells, by expression of hSLIM nucleic acid molecules, or by direct chemical synthesis.

hSLIM Polypeptide Purification

An hSLIM polypeptide can be purified from any human cell which expresses the polypeptide, including human host cells which have been transfected with hSLIM nucleic acid molecules. A purified hSLIM polypeptide is separated from other compounds that normally associate with the hSLIM polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

An enzymatically active hSLIM polypeptide (i.e., an hSLIM polypeptide with E3 ligase activity) can be prepared by purifying and/or refolding the polypeptide in the presence of zinc and/or in the absence of a zinc chelator such as EDTA, DTPA, TPEN, and EGTA.

A preparation of purified hSLIM polypeptides is at least 80% pure; preferably, the preparations are 90%, 95%, or 99% pure. Purity of the preparations can be assessed by any means known in the art, such as SDS-polyacrylamide gel electrophoresis.

Expression of hSLIM Nucleic Acid Molecules

To express an hSLIM nucleic acid molecule, the nucleic acid molecule can be inserted into an expression vector which contains the necessary elements for the transcription and translation of the inserted coding sequence. Methods which are well known to those skilled in the art can be used to construct expression vectors containing sequences encoding hSLIM polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989; these references are incorporated in their entireties herein.

A variety of expression vector/host systems can be utilized to contain and express sequences encoding an hSLIM polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems, particularly mammalian systems, including human systems. See WO 01/98340, which is incorporated herein by reference in its entirety.

Host cells

Host cells can be, for example, bacterial, insect, mammalian, or human cells. A host cell strain can be chosen for its ability to modulate the expression of the inserted sequences or to process the expressed hSLIM polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138) are available from the American Type Culture Collection (ATCC; 10801 University Boulevard, Manassas, Va. 20110-2209) and can be chosen to ensure the correct modification and processing of the foreign protein. See WO 01/98340.

Alternatively, host cells which contain an hSLIM nucleic acid molecule and which express an hSLIM polypeptide can be identified by a variety of procedures known to those of skill in the art. Examples include enzyme-linked immunosorbent assay (ELISA), radioimmunoassay (RIA), and fluorescence activated cell sorting (FACS). Hampton et al., SEROLOGICAL METHODS: A LABORATORY MANUAL, APS Press, St. Paul, Minn., 1990) and Maddox et al., J. Exp. Med. 158, 1211-1216, 1983), which are incorporated herein by reference in their entireties. See also WO 01/98340.

A wide variety of labels and conjugation techniques are known by those skilled in the art and can be used in various nucleic acid and amino acid assays. Methods for producing labeled hybridization or PCR probes for detecting sequences related to nucleic acid molecules encoding hSLIM polypeptides include oligolabeling, nick translation, end-labeling, or PCR amplification using a labeled nucleotide. Alternatively, sequences encoding an hSLIM polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of hSLIM Polypeptides

Host cells transformed with nucleotide sequences encoding an hSLIM polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing nucleic acid molecules which encode hSLIM polypeptides can be designed to contain signal sequences which direct secretion of soluble hSLIM polypeptides through a prokaryotic or eukaryotic cell membrane or which direct the membrane insertion of membrane-bound hSLIM polypeptide. See WO 01/98340.

Chemical Synthesis of hSLIM Polypeptides

An hSLIM polypeptide can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995), which is incorporated herein by reference in its entirety. Protein synthesis can be performed using manual techniques or by automation. Automated synthesis can be achieved, for example, using Applied Biosystems 431A Peptide Synthesizer (Perkin Elmer). Optionally, fragments of hSLIM polypeptides can be separately synthesized and combined using chemical methods to produce a full-length molecule. See WO 01/98340.

As will be understood by those of skill in the art, it may be advantageous to produce hSLIM polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter hSLIM polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies Which Specifically Bind to hSLIM Polypeptides

Any type of antibody known in the art can be generated to bind specifically to an epitope of an hSLIM polypeptide. The term “antibody” includes intact immunoglobulin molecules, as well as fragments thereof which are capable of binding an antigen. These include hybrid (chimeric) antibody molecules (e.g., Winter et al., Nature 349, 293-99, 1991; U.S. Pat. No. 4,816,567); F(ab′)₂ and F(ab) fragments and Fᵥ molecules; non-covalent heterodimers (e.g., Inbar et al., Proc. Natl. Acad. Sci. U.S.A. 69, 2659-62, 1972; Ehrlich et al., Biochem 19, 4091-96, 1980); single-chain Fv molecules (sFv) (e.g., Huston et al., Proc. Natl. Acad. Sci. U.S.A. 85, 5897-83, 1988); dimeric and trimeric antibody fragment constructs; minibodies (e.g., Pack et al., Biochem 31, 1579-84, 1992; Cumber et al., J. Immunology 149B, 120-26, 1992); humanized antibody molecules (e.g., Riechmann et al., Nature 332, 323-27, 1988; Verhoeyan et al., Science 239, 1534-36, 1988; and U.K. Patent Publication No. GB 2,276,169, published 21 Sep. 1994); and any functional fragments obtained from such molecules, as well as antibodies obtained through non-conventional processes such as phage display. See See WO 01/98340. Each of these references is incorporated herein in its entirety. Preferably, the antibodies are monoclonal antibodies. Methods of obtaining monoclonal antibodies are well known in the art. Intracellular antibodies (“intrabodies”) are preferred.

Typically, at least 6, 8, 10, or 12 contiguous amino acids are required to form an epitope. However, epitopes which involve non-contiguous amino acids may require more, e.g., at least 15, 25, or 50 amino acids. Preferably, an hSLIM antibody does not bind to the PDZ or LIM domains.

hSLIM antibodies can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Antibodies of the invention can comprise a detectable label, such an enzymatic, fluorescent, luminescent, isotopic, or affinity label.

Typically, an antibody that specifically binds to an hSLIM polypeptide provides a detection signal at least 5-, 10-, or 20-fold higher than a detection signal provided with other proteins when used in an immunochemical assay. Preferably, antibodies that specifically bind to hSLIM polypeptides do not detect other proteins in immunochemical assays and can immunoprecipitate an hSLIM polypeptide from solution.

hSLIM Antisense Oligonucleotides

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of hSLIM gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990, incorporated herein by reference in their entireties.

Modifications of hSLIM gene expression can be obtained by designing antisense oligonucleotides that will form duplexes to the control, 5′, or regulatory regions of the hSLIM gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994, which is incorporated herein by reference in its entirety). An antisense oligonucleotide also can be designed to block translation of mRNA by preventing the transcript from binding to ribosomes. See WO 01/98340.

hSLIM siRNA Molecules

siRNA molecules (“small interfering” or “short interfering” RNA) are described, for example, in US 2004/0235171, which is incorporated by reference herein in its entirety. An hSLIM siRNA molecule according to the invention is an RNA duplex of nucleotides which is targeted to the hSLIM gene. The duplex is the structure formed by the complementary pairing between two regions of an RNA molecule. The targeting occurs because the nucleotide sequence of the duplex portion of the siRNA is complementary to a nucleotide sequence of the hSLIM gene.

In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

Preferred hSLIM siRNA molecules are shown in SEQ ID NOS:15 and 16.

Screening Methods

The invention provides assays for screening test compounds for their ability to affect hSLIM function, including the ability of hSLIM to bind to a STAT protein such as STAT1 or STAT4, E3 ligase activity, and for their ability to affect downstream functions of hSLIM such as STAT phosphorylation, STAT-mediated transcription, IFNγ production, Th1 or Th2 cell differentiation, and T-bet activity.

A test compound which increases an hSLIM binding activity or an hSLIM functional activity is a potential therapeutic agent for treating asthma, allergic rhinitis, and chronic viral infections, as well as autoimmune disorders, such as systemic lupus erythematosus; rheumatoid arthritis; myasthenia gravis, multiple sclerosis, type I diabetes mellitus, Sjögren's syndrome Goodpasture's syndrome; Grave's disease; Hashimoto's thyroiditis; pemphigus vulgaris; scleroderma; autoimmune hemolytic anemia; autoimmune thrombocytopenic purpura; polymyositis and dermatomyositis; pernicious anemia; ankylosing spondylitis; vasculitis, inflammatory bowel disease, ulcerative colitis, Crohn's disease.

A test compound which decreases an hSLIM binding activity or an hSLIM functional activity is a potential therapeutic agent for treating malignancies (e.g., acute lymphoblastic leukemia; acute myeloid leukemia; adrenocortical carcinoma; AIDS-related lymphoma; cancer of the bile duct; bladder cancer; bone cancer; breast cancer; bronchial adenomas; carcinoid tumors; adrenocortical carcinoma; central nervous system lymphoma; cervical cancer; colon cancer; colorectal cancer; cutaneous T-cell lymphoma; B-cell lymphoma; endometrial cancer; vaginal cancer; epithelial cancer; endometrial cancer; intraocular melanoma; retinoblastoma; hairy cell leukemia; liver cancer; osteosarcoma; malignant fibrous histiocytoma; brain stem glioma,; brain tumor; Hodgkin's disease; lung cancer; non-Hodgkin's lymphoma; melanoma; multiple myeloma; neuroblastoma; prostate cancer; retinoblastoma; acute lymphoblastic leukemia; Ewing's sarcoma; Kaposi's sarcoma; Waldenstrom's macroglobulinemia; Wilm's tumor) and transplantation and graft rejection.

Test compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409). Each of these references is incorporated herein by reference in its entirety.

High Through-Put Screening

Screening methods of the invention can be used in high through-put screening formats. Using high throughput screening, many discrete compounds can be tested in parallel so that large numbers of test compounds can be quickly screened. The most widely established techniques utilize 96-well microtiter plates, however 384- or 1536- plates also can be used. As is known in the art, a variety of instruments, materials, pipettors, robotics, plate washers, and plate readers are commercially available.

Binding Assays

In binding assays, either the test compound or the hSLIM polypeptide (e.g., wild-type hSLIM or WT-Δ52-hSLIM as shown in SEQ ID NO:4) can comprise a detectable label, such as a fluorescent, radioisotopic, chemiluminescent, or enzymatic label, such as horseradish peroxidase, alkaline phosphatase, or luciferase. Detection of a test compound that is bound to the hSLIM polypeptide can then be accomplished, for example, by direct counting of radioemmission, by scintillation counting, or by determining conversion of an appropriate substrate to a detectable product.

Alternatively, binding of a test compound to an hSLIM polypeptide can be determined without labeling either of the interactants. For example, a microphysiometer can be used to detect binding of a test compound with an hSLIM polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and an hSLIM polypeptide (McConnell et al., Science 257, 1906-1912, 1992).

Determining the ability of a test compound to bind to an hSLIM polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an hSLIM polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent W094/10300), to identify other proteins which bind to or interact with the hSLIM polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding an hSLIM polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein that interacts with the hSLIM polypeptide.

It may be desirable to immobilize either the hSLIM polypeptide (or nucleic acid molecule) or the test compound to facilitate separation of bound from unbound forms of one or both of the interactants, as well as to accommodate automation of the assay. Thus, either the hSLIM polypeptide (or nucleic acid molecule) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the enzyme polypeptide (or polynucleotide) or test compound to a solid support, including use of covalent and non-covalent linkages, passive absorption, or pairs of binding moieties attached respectively to the polypeptide (or nucleic acid molecule) or test compound and the solid support. Test compounds are preferably bound to the solid support in an array, so that the location of individual test compounds can be tracked. Binding of a test compound to an hSLIM polypeptide (or nucleic acid molecule) can be accomplished in any vessel suitable for containing the reactants. Examples of such vessels include microtiter plates, test tubes, and microcentrifuge tubes.

In one embodiment, the hSLIM polypeptide is present in a fusion protein comprising a domain which allows the hSLIM polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed hSLIM polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either an hSLIM polypeptide (or nucleic acid molecule) or a test compound can be immobilized utilizing conjugation of biotin and streptavidin. Biotinylated hSLIM polypeptides (or nucleic acid molecules) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to an hSLIM polypeptide but which do not interfere with a desired binding site, such as the E3 ligase active site of the hSLIM polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the hSLIM polypeptide or test compound, enzyme-linked assays which rely on detecting an activity of the hSLIM polypeptide, and SDS gel electrophoresis under non-reducing conditions.

Screening for test compounds which bind to an hSLIM polypeptide or nucleic acid molecule also can be carried out in an intact cell. Any cell which comprises an hSLIM polypeptide or nucleic acid molecule (either naturally occurring or introduced) can be used in a cell-based assay system. Binding of the test compound to an hSLIM polypeptide or nucleic acid molecule can be determined as described above.

E3 Ligase Activity

Ubiquitination of a substrate protein occurs through a three-step process. First, ubiquitin is activated by a ubiquitin activating enzyme, E1, and is then transferred to a ubiquitin conjugating enzyme, E2. The activated ubiquitin is then attached to the target protein by an E3 ubiquitin ligase enzyme. In the case of Ring-containing E3 ligases, such as hSLIM, the E3 ligase itself also becomes ubiquitinated.

Thus, in some embodiments of the invention, test compounds are tested for the ability to increase or decrease the E3 ligase activity of an hSLIM polypeptide. A test compound preferably increases or decreases E3 ligase activity of an hSLIM polypeptide by at least about 10, preferably about 50, more preferably about 75, 90, or 100% relative to the E3 ligase activity of the hSLIM polypeptide in the absence of the test compound.

E3 ligase activity can be measured by any means known in the art. For example, 1-Fluorescence Resonance Energy Transfer (FRET) can be used to measure ubiquitin transfer in cell free assays. In one embodiment the assay detects ubiquitinated hSLIM; in another embodiment the assay detects the ability of hSLIM to ubiquitinate a STAT protein, such as STAT1 or STAT4.

In both FRET assay formats, ubiquitin is prelabeled with biotin and is transferred by E1 and E2 to hSLIM. When the aim is to detect the ubiquitination of hSLIM, tagged-hSLIM, such as GST-hSLIM or HA-hSLIM, is used in the assay. Tagged hSLIM is incubated with E1 and E2 in the presence of ATP and biotin-ubiquitin. Compounds or controls are added to the reaction. At the termination of the enzymatic reaction, the Bio-Ub-Tagged-hSLIM is preincubated with allophycocyanin-labeled streptavidin (APC-SA) followed by anti-tag antibody (such as anti-GST or anti-HA, respectively) labeled with LANCE europium (Eu3+) chelate. When ubiquitination is present, Eu3+and APC are brought into close proximity, permitting energy transfer between the two fluorescent labels.

Alternatively, tagged-STAT, such as His-STAT1 or his-STAT4, is incubated with E1, E2 and hSLIM along with biotin-ubiquitin. Bio-Ub-STAT is detected using allophycocyanin-labeled streptavidin (APC-SA) and anti-tag antibody (such as anti-his) labeled with LANCE Eu3+. FRET is measured with excitation of Eu3+ at 340 nm and time-resolved fluorescence at the emission wavelength of APC at 665 nm.

Ubiquitin transfer also can be measured using allophycocyanin-labeled streptavidin (APC-SA) and anti-tag antibody labeled with LANCE Eu3+ is used for a DELFIA assay read-out.

An alternative approach to FRET or DELFIA is a proximity assay also known as Alpha screen, the tagged-hSLIM protein is ubiquitinated as described above. The ubiquitinated tagged-hSLIM is captured by anti-tag acceptor and streptavidin donor beads. This proximity of acceptor and donor beads induced by the simultaneous binding to ubiquitinated tagged-hSLIM allows the generation of the AlphaScreen signal.

Transcriptional Activity of STAT Proteins

Cell-based reporter assays can be used to measure the transcriptional activity of a STAT protein, such as STAT1 or STAT4.

STAT1 and STAT4 are transcription factors that, upon phosphorylation and activation, translocate to the nucleus and bind to consensus sites on specific promoters, thereby regulating gene expression. These properties have been used to develop a cell based reporter assay. The STAT binding site, namely interferon-γ activated sequence (GAS) site, has been cloned in 6 repeats upstream of the Photinus pyralis (firefly) luciferase gene in a reporter plasmid. Transcriptional activation of STAT1 or STAT4 enhances luciferase expression. Regulation of STAT protein by hSLIM is reflected by the amount of GAS-Luc produced. Luciferase expression is quantitated using a luminometer, and directly correlates with light emission.

This assay system was used to generated data shown in the Examples below and can be readily modified for a high through-put assay system. Cells grown in suspension can be transfected with the appropriate constructs and placed in multi-well plates, such as 96, 384 or 1536 well plates. The cells are stimulated with interferon in the presence or absence of compounds, lysed, and evaluated for luciferase expression using an add-only format.

hSLIM Gene Expression

In another embodiment, test compounds are screened for the ability to affect hSLIM gene expression. An hSLIM nucleic acid molecule is contacted with a test compound, and the expression of an RNA or polypeptide product of the nucleic acid molecule is determined. The level of expression of appropriate mRNA or polypeptide in the presence of the test compound is compared to the level of expression of mRNA or polypeptide in the absence of the test compound. The test compound can then be identified as a modulator of expression based on this comparison.

Such screening can be carried out either in a cell-free assay system or in an intact cell. Any cell which expresses an hSLIM nucleic acid molecule can be used in a cell-based assay system. The hSLIM nucleic acid molecule can be naturally occurring in the cell or can be introduced using techniques such as those described above. Either a primary culture or an established cell line can be used.

The level of hSLIM mRNA or polypeptide expression can be determined by methods well known in the art for detecting mRNA or polypeptide. Either qualitative or quantitative methods can be used. The presence of polypeptide products of an hSLIM nucleic acid molecule can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into an hSLIM polypeptide.

IFNγ Production

IFNγ production can be measured as disclosed, for example, in Example 7.

Th1 or Th2 Cell Differentiation

In vitro Assays for Th1 activities are disclosed, for example, in U.S. Pat. No. 6,657,055; Sundrud et al., J. Immunol. 171, 3542-49, 2003; Shirota et al., J. Immunol. 173, 5002-07, 2004; and Szabo et al., Annu. Rev. Immunol. 21, 713-58, 2003, which are incorporated herein by reference in their entireties.

T-Bet Activity

The effect of hSLIM modulation on T-bet activity can be assessed using methods well known in the art. See Afkarian et al., Nature Immunol. 3, 549-57, 2002; Lametschwandtner et al., J. Allergy Clin. Immunol. 113, 987-94, 2004, which are incorporated herein by reference in their entireties.

hSLIM Pharmaceutical Compositions

The invention also provides pharmaceutical compositions which can be administered to a patient to treat a variety of disorders such as those disclosed above. Pharmaceutical compositions of the invention can comprise, for example, an hSLIM polypeptide, an hSLIM nucleic acid molecule, hSLIM antisense oligonucleotides, siRNAs which affect hSLIM gene expression, antibodies which specifically bind to an hSLIM polypeptide, or modulators of an hSLIM polypeptide activity identified by the methods described above. Pharmaceutical compositions also can comprise additional therapeutic agents, including immunomodulatory agents such as vaccines, dendritic cells, monoclonal antibodies, and the like.

The compositions can be administered alone or in combination with at least one other agent, such as stabilizing compound, which can be administered in any sterile, physiologically acceptable pharmaceutical vehicle, including, but not limited to, saline, buffered saline, dextrose, and water. Typically such vehicles are non-pyrogenic. The compositions can be administered to a patient alone, or in combination with other therapeutic agents.

In addition to the active ingredients, these pharmaceutical compositions can contain suitable pharmaceutically acceptable carriers comprising excipients and auxiliaries which facilitate processing of the active compounds into preparations which can be used pharmaceutically. Such ingredients are well known in the art. See, e.g., 01/98340 and REMINGTON'S PHARMACEUTICAL SCIENCES (Maack Publishing Co., Easton, Pa.), which are incorporated herein by reference in their entireties.

After pharmaceutical compositions have been prepared, they can be placed in an appropriate container and labeled for treatment of an indicated condition. Such labeling would include amount, frequency, and method of administration.

Pharmaceutical compositions of the invention can be administered by any number of routes including, but not limited to, oral, pulmonary, intranodal, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

### Cloning of Human SLIM

Full length WT-SLIM: cDNA coding for human SLIM was cloned by performing one-step RT-PCR on human testis total RNA using the SUPERSCRIPT III™ RT-PCR Kit (Invitrogen). Gene-specific primers for hSLIM used have the following sequences:

(SEQ ID NO: 11)

5′:
TTAAGAATTCGCCACCATGGCGTTGACGGTGGATGTGGCCGGGCCAGC

(SEQ ID NO: 12)

3′:
TTAAGCGGCCGCTCAGGCCCGAGAGCTGAGGGTGGCAGGTGC

The 1 Kb PCR product was then directed subcloned into the mammalian expression vector pcDNA3.1 (Invitrogen) and the bacterial expression vector pGEX (Amersham). Sequence analysis and alignment reveal that this cDNA species (SEQ ID NO:1) encodes an open reading frame of 352 amino acid residues (SEQ ID NO:2), identical to that of an entry in the GeneBank BC021556 and NP067643 (SEQ ID NO:2) for Homo sapiens PDZ and LIM domain 2 (mystique), transcript variant 2. There is only one base pair difference between the two sequences. However, amino acid sequences are identical.

### EXAMPLE 2

### Reverse Transcription PCR and Isoform Identification

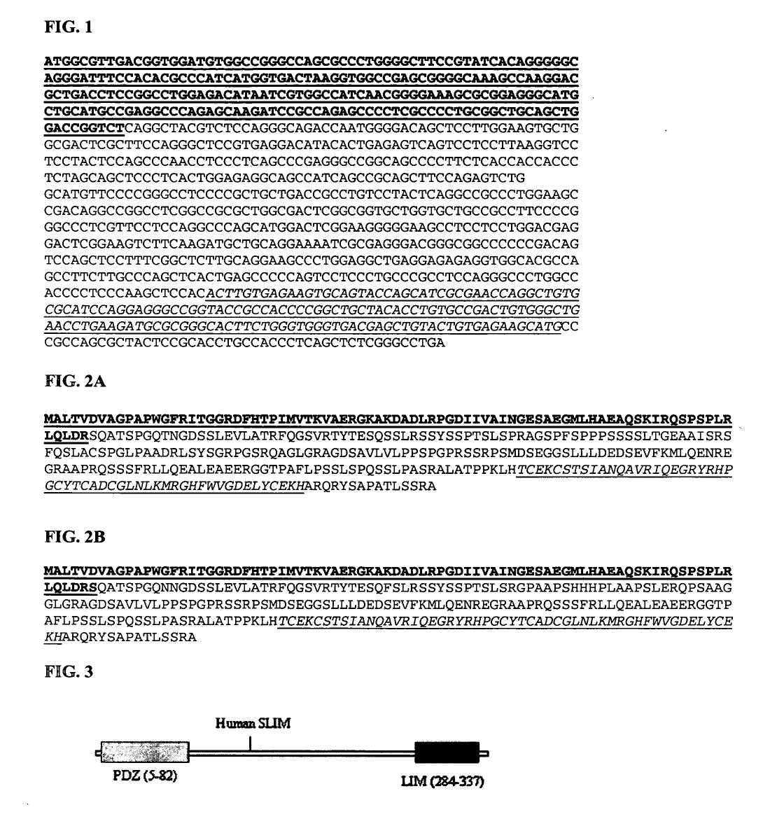

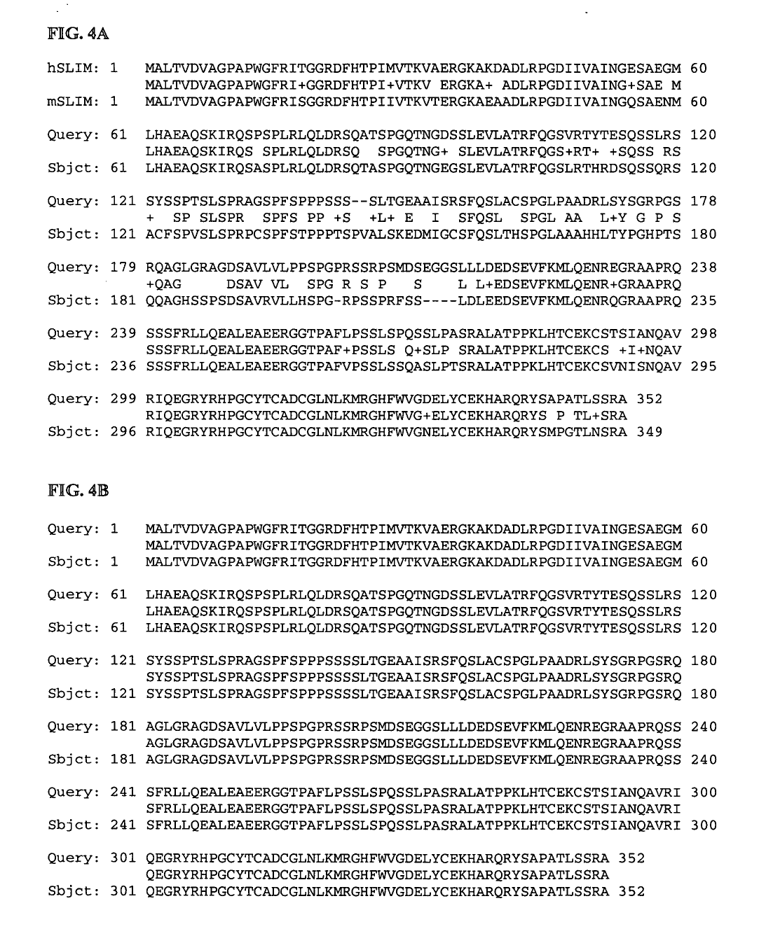

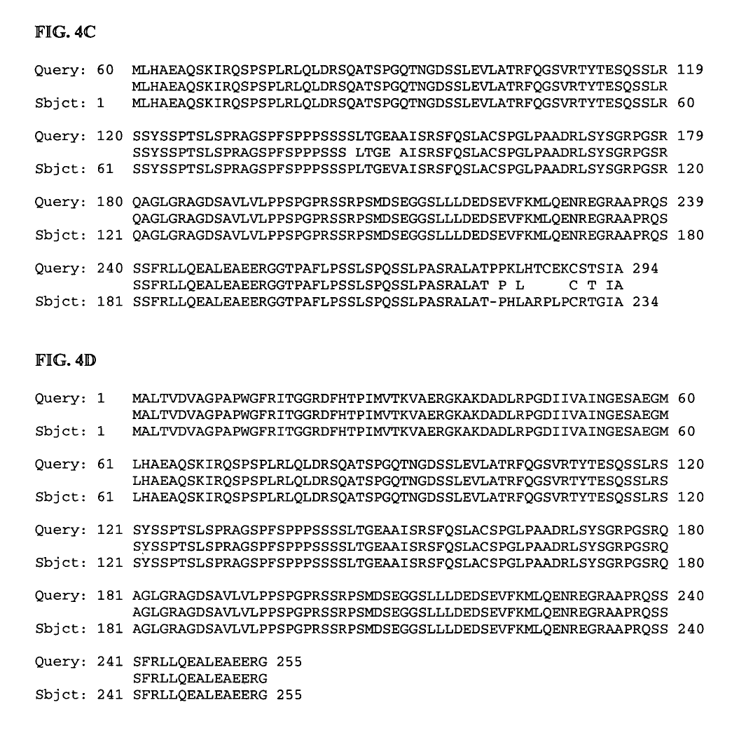

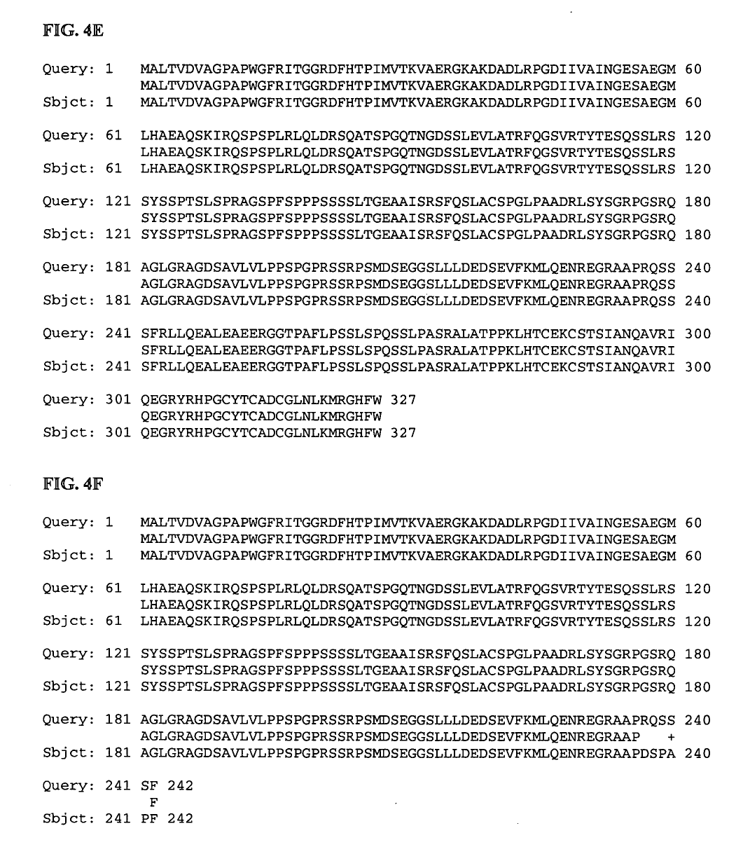

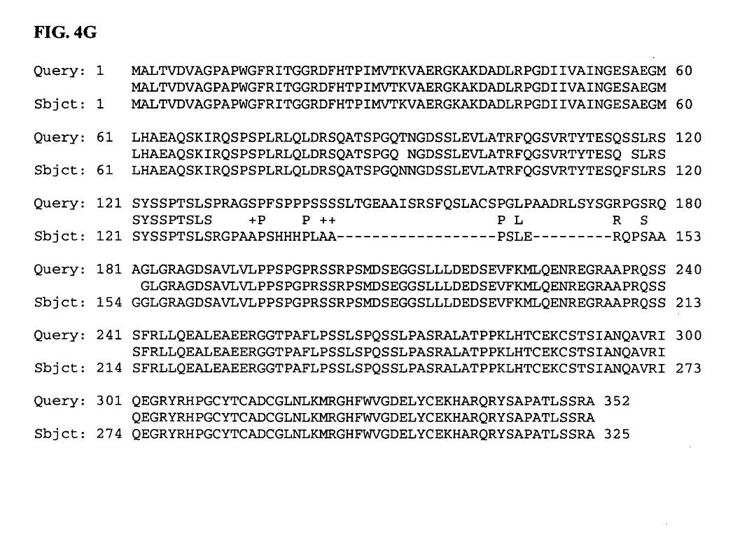

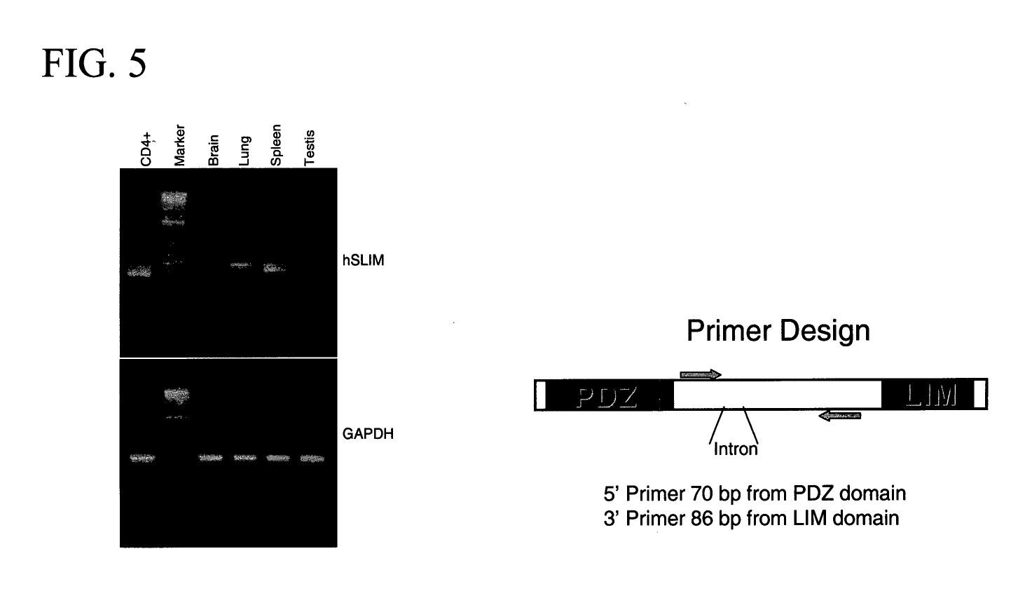

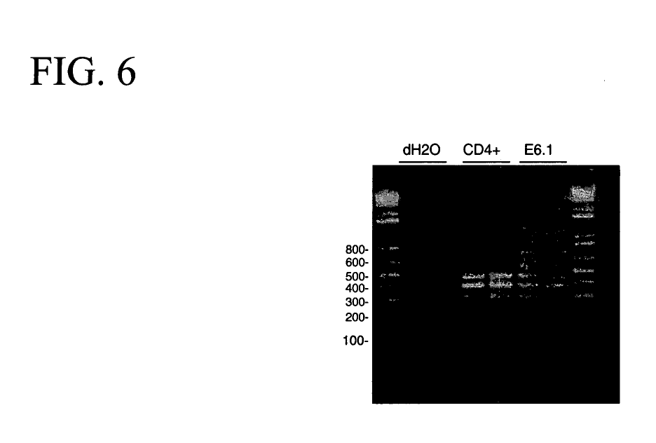

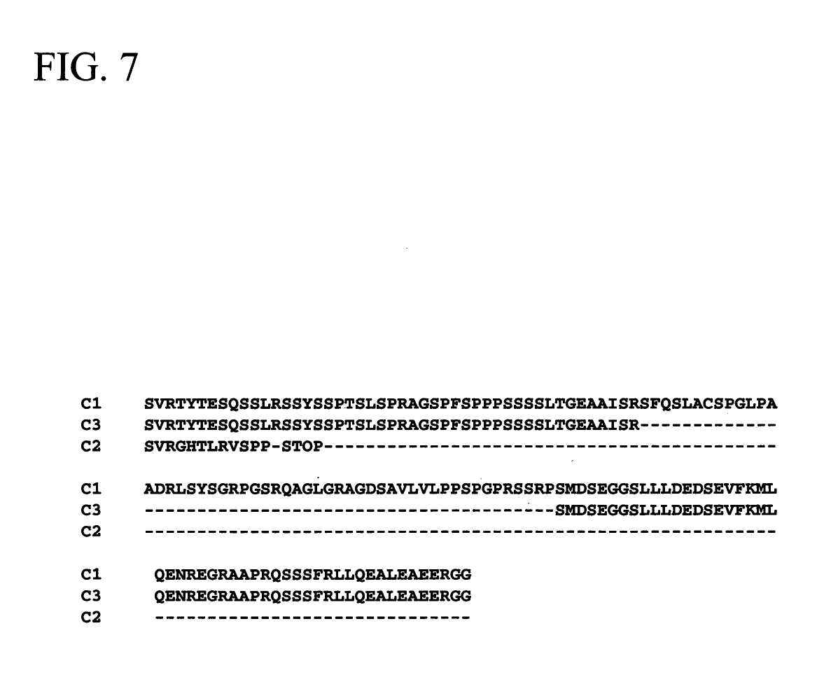

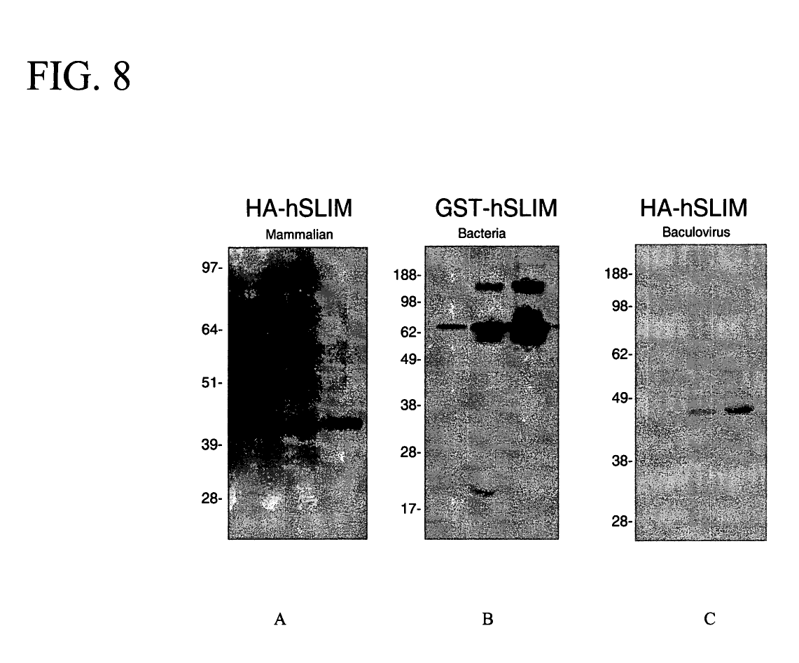

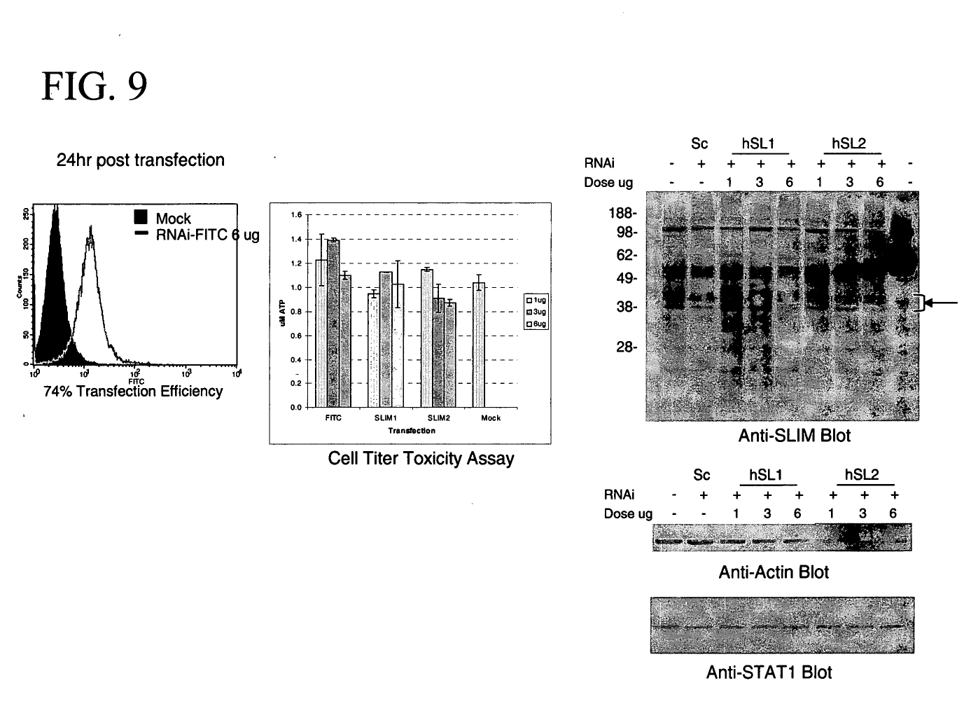

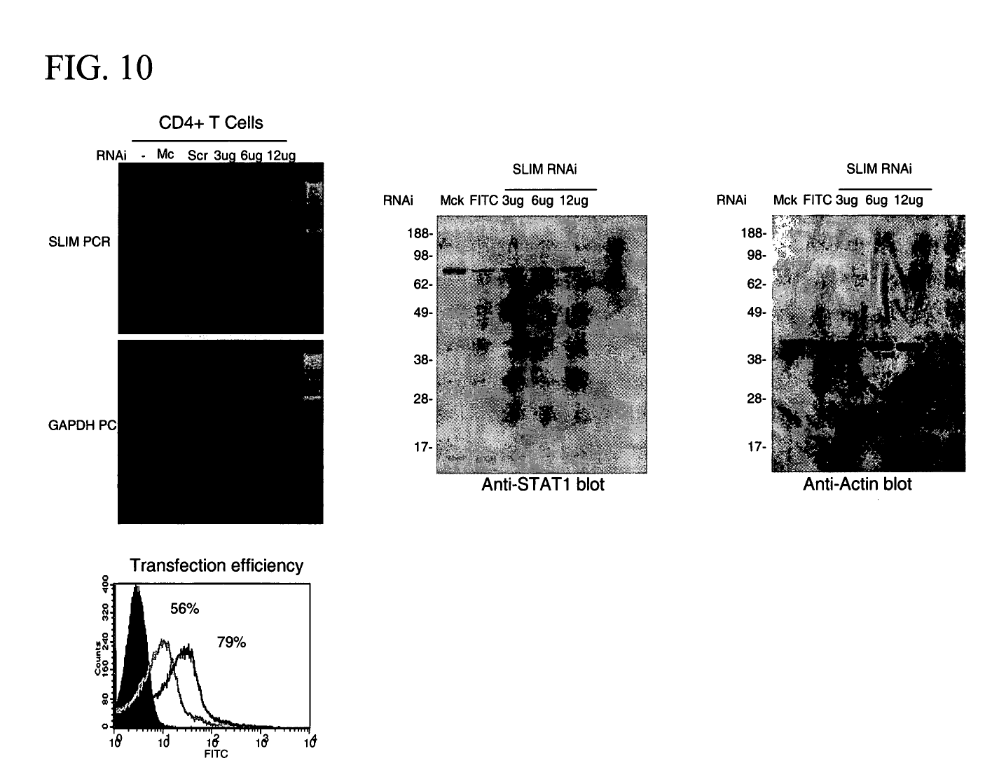

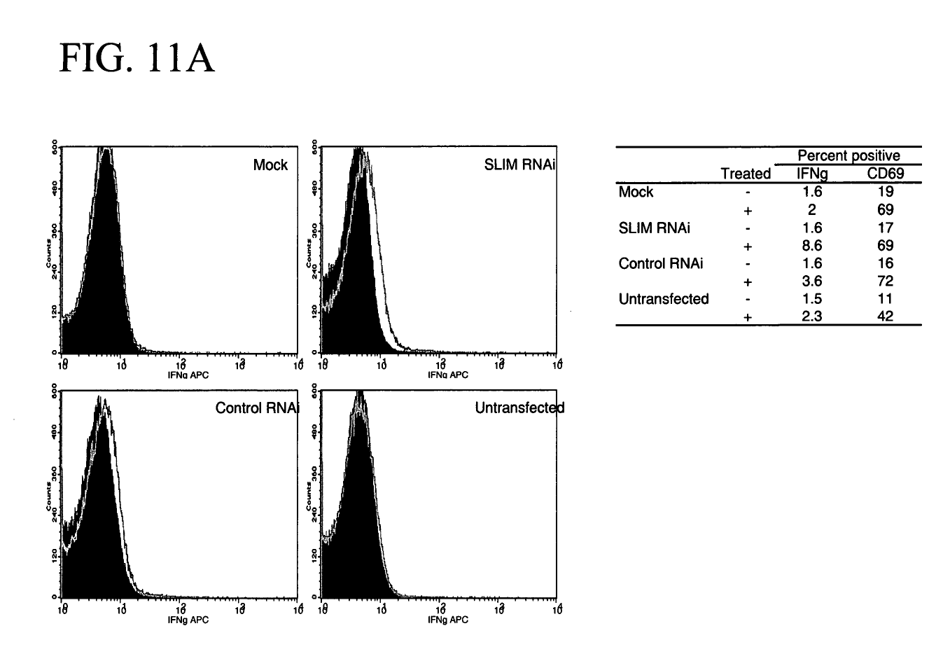

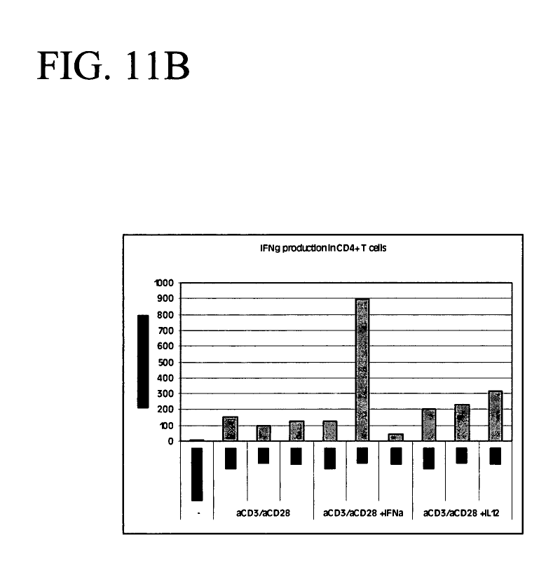

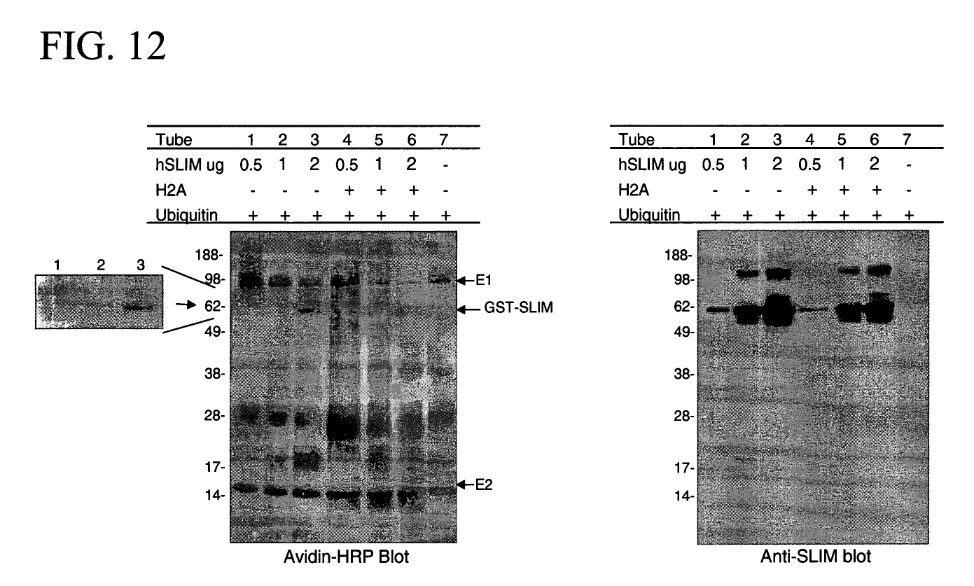

The human SLIM cDNA sequence contains 2 conserved domains found in several protein families: a PDZ domain at its N-terminus and a LIM domain at its C-terminus (see FIG. 1). In order to amplify SLIM-specific cDNA, we designed PCR primers that avoid these domains by locating the primers 3′ of the PDZ motif and 5′ to the LIM domain (FIG. 3). The sequences of human SLIM primers were:

| | |
|---|---|
| 5′: CCGTGAGGACATACACTGAGAGTCA | (SEQ ID NO: 13) |
| 3′: CACCTCTCTCCTCAGCCTCCAG | (SEQ ID NO: 14) |

Cells were harvested 24-72 hr post-transfection and TRIZOL® extraction (Invitrogen) was performed to isolate total RNA. Equivalent amounts of total RNA were reverse-transcribed into cDNA using SUPERSCRIPT III™ reverse-transcriptase, oligo-dT and First Strand cDNA synthesis kit (Invitrogen). SLIM-specific PCR was performed using 10 pM of forward and reverse primers and PLATINUM® Taq DNA polymerase (Invitrogen) in 30-32 cycles of amplification. Glyceraldehyde-3-phosphate dehydrogenase (GAPDH) primers were used as control. PCR products were separated on a 2% agarose gel and stained with ethidium bromide.

A similar approach was used for isoform identification. In particular, total RNA from human brain, lung, spleen and testis were purchased from Biochain. The PCR reaction was performed as described above. The PCR products were separated on an agarose gel, the individual bands extracted, cloned and individual clones were sequence verified. Specific isoforms were subcloned into pGEX6 expression vectors (Amersham) and verified for protein expression.

SLIM Isoform WT-Δ52-hSLIM: An isoform predominantly expressed in CD4+T cells was WT-Δ52-hSLIM (SEQ ID NO:4), which lacks 52 amino acids relative to full length WT-hSLIM, starting at aa 154. To generate recombinant WT-Δ52-hSLIM, WT-hSLIM was amplified by PCR and an Afl II/Nru I fragment of the RT-PCR product was used to substitute the corresponding section in WT-hSLIM.

### EXAMPLE 3

### Generation of hSLIM Point Mutant

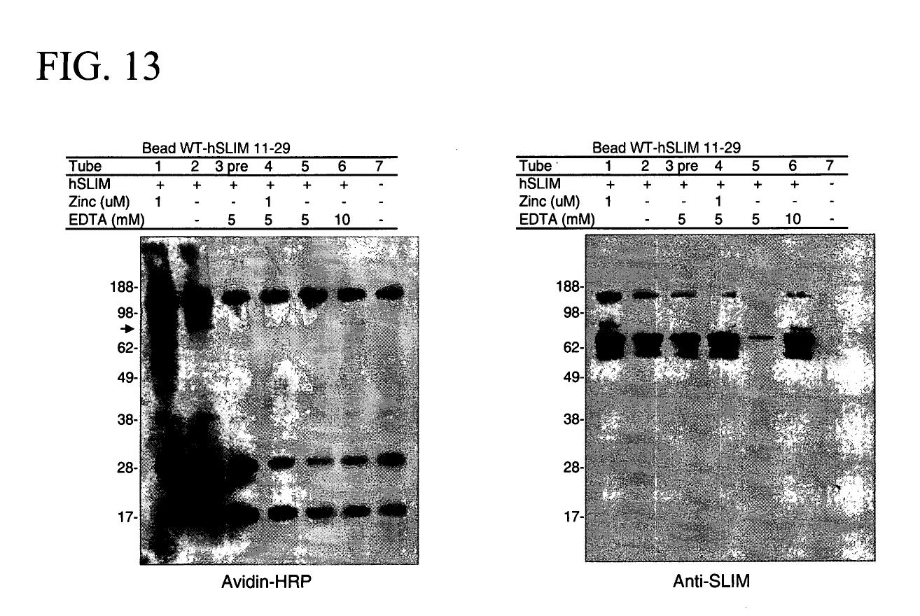

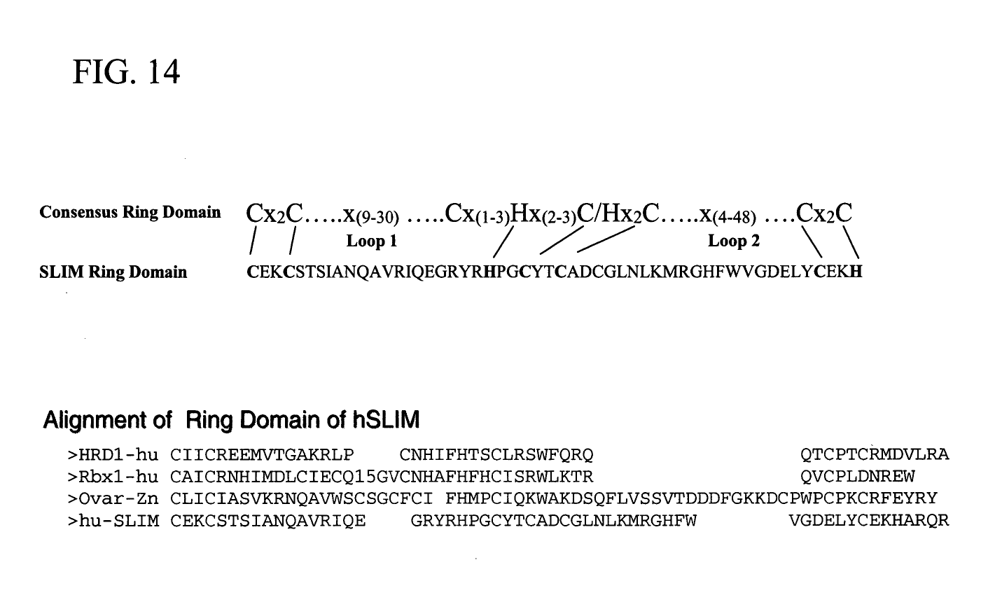

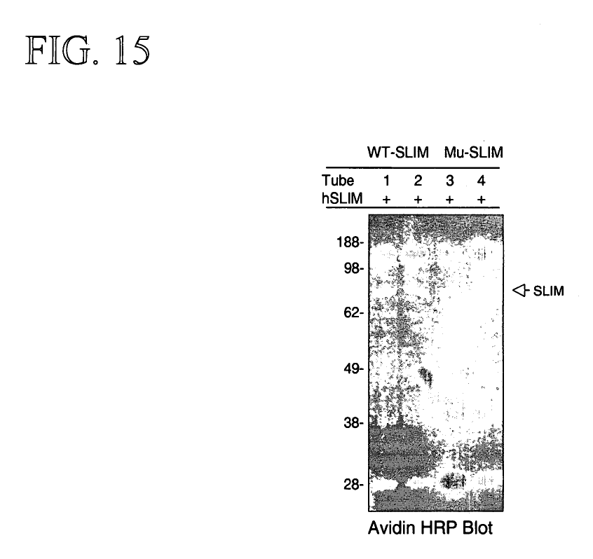

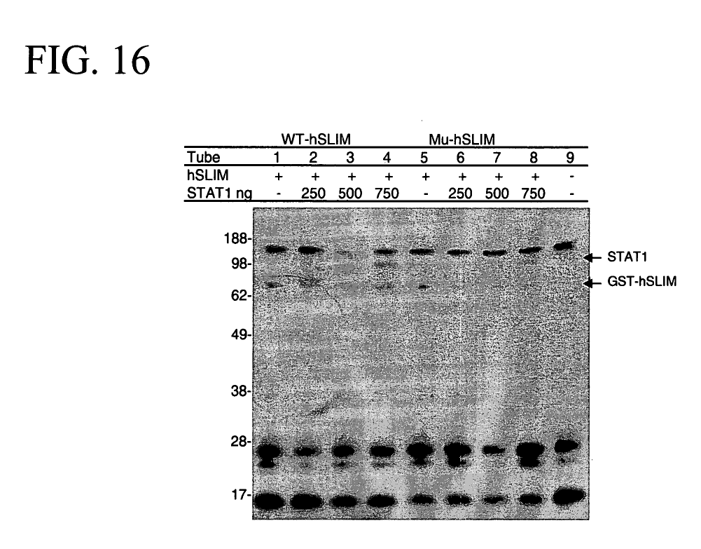

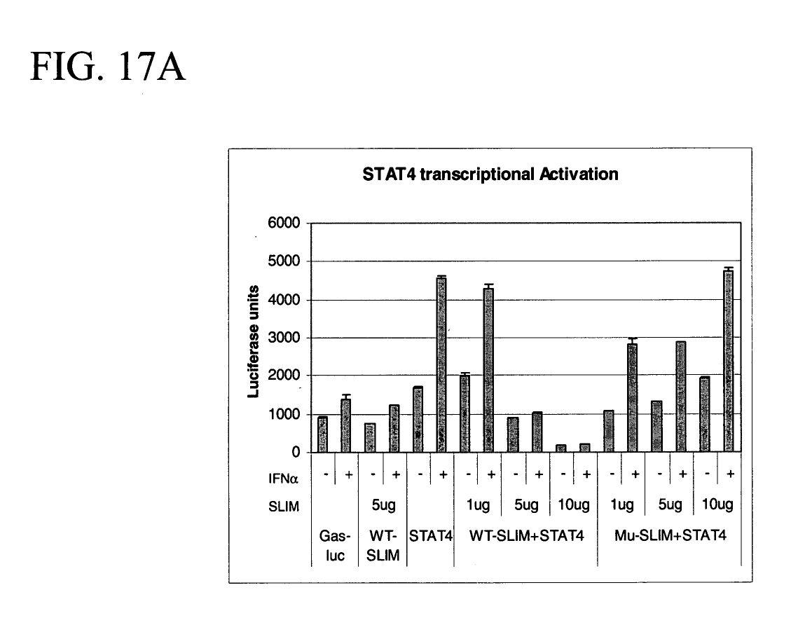

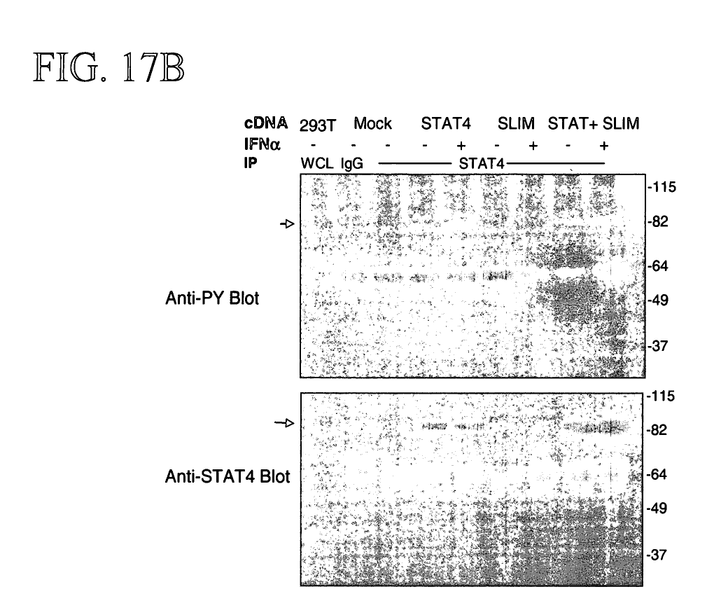

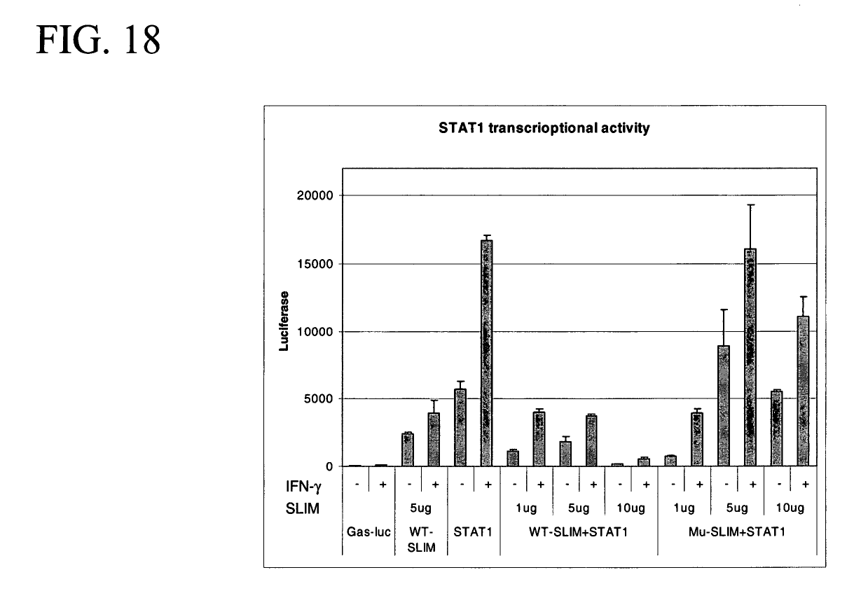

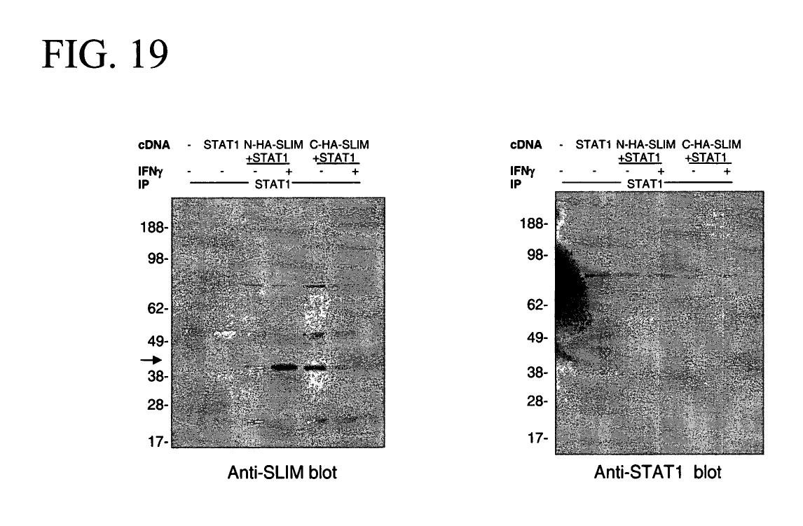

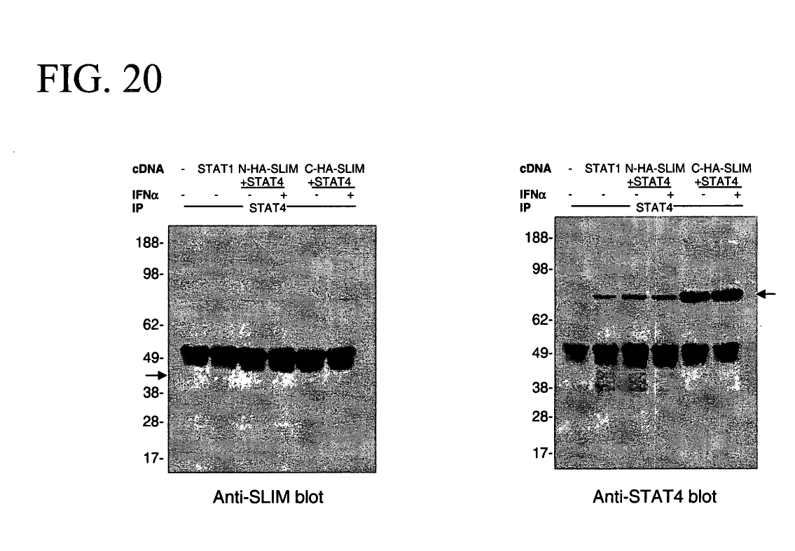

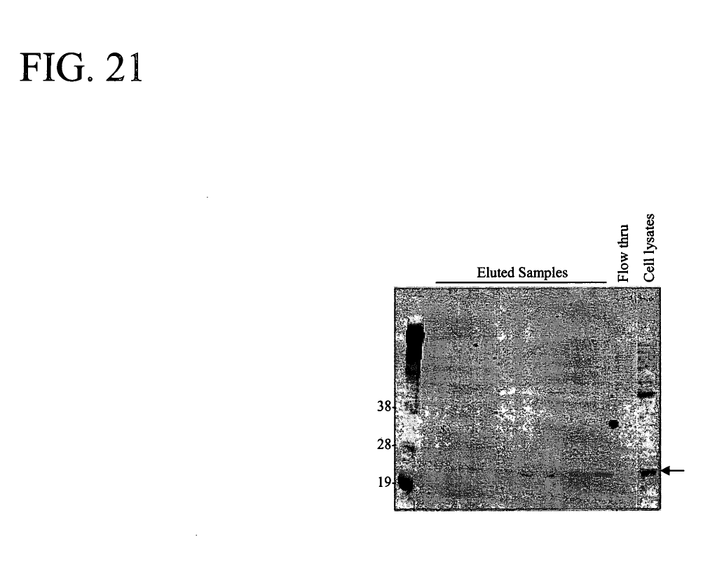

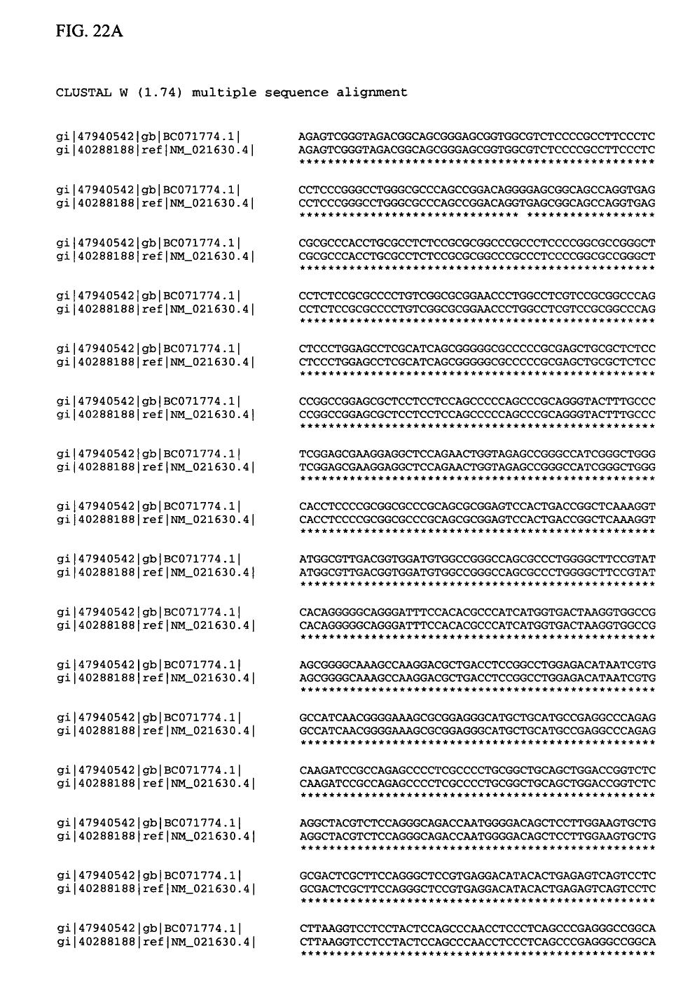

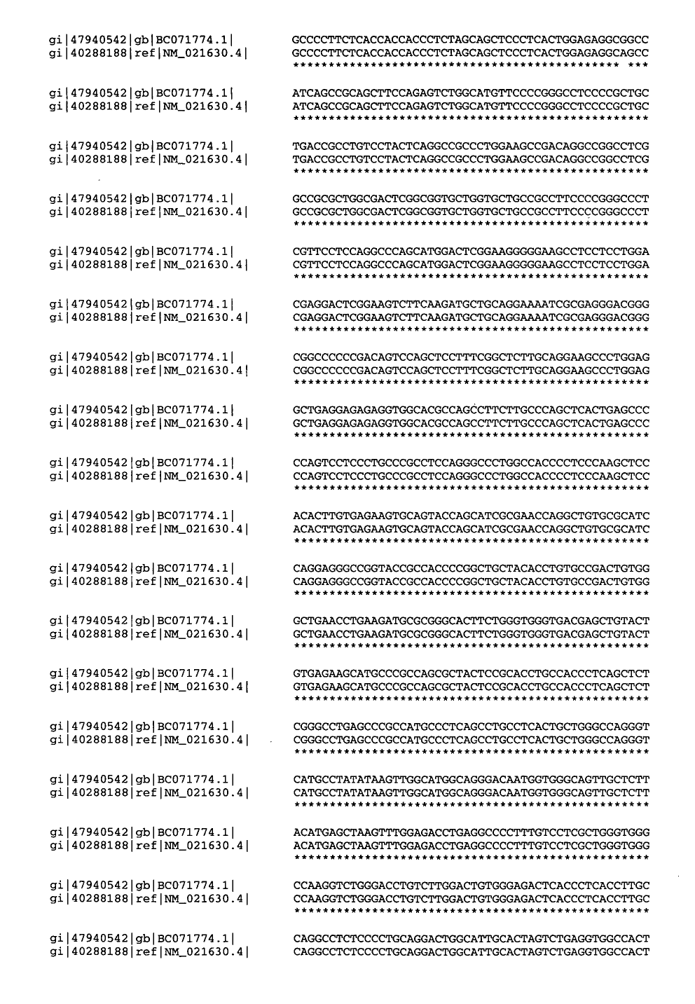

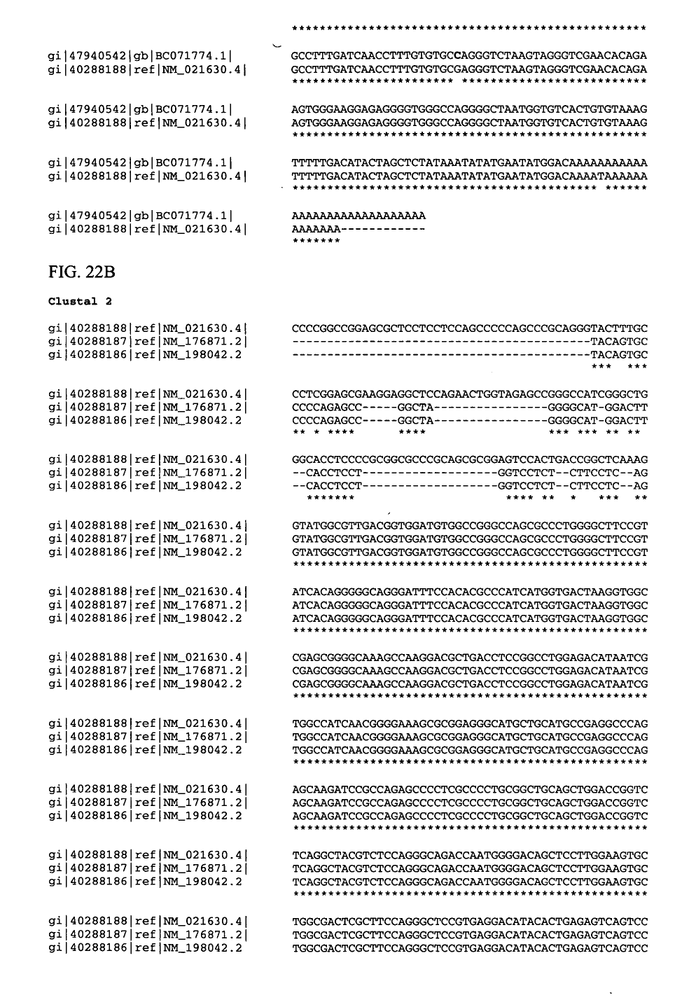

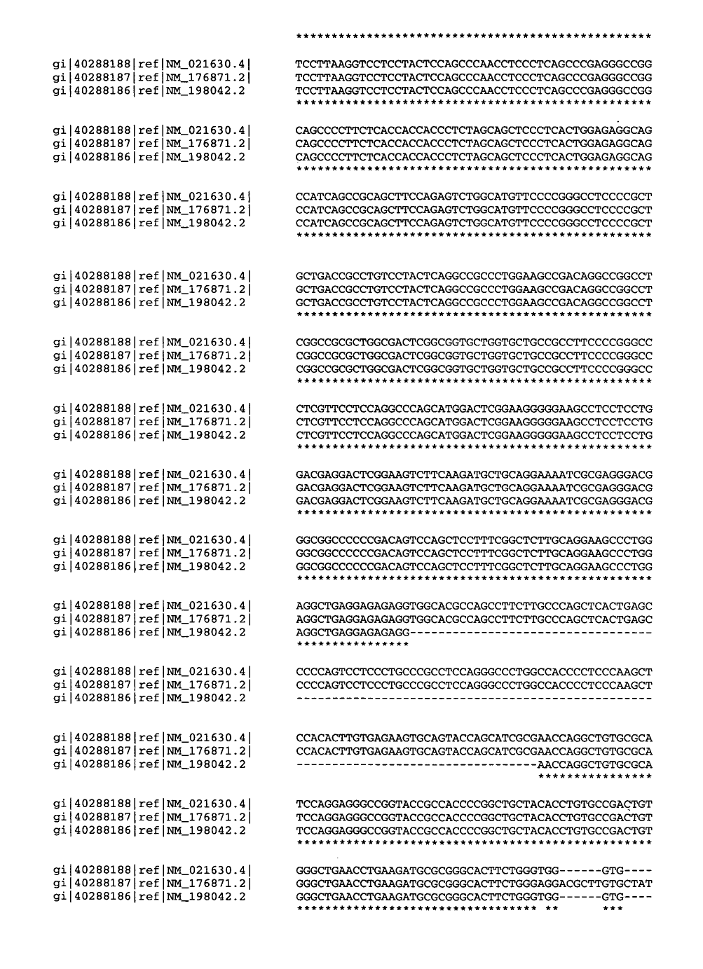

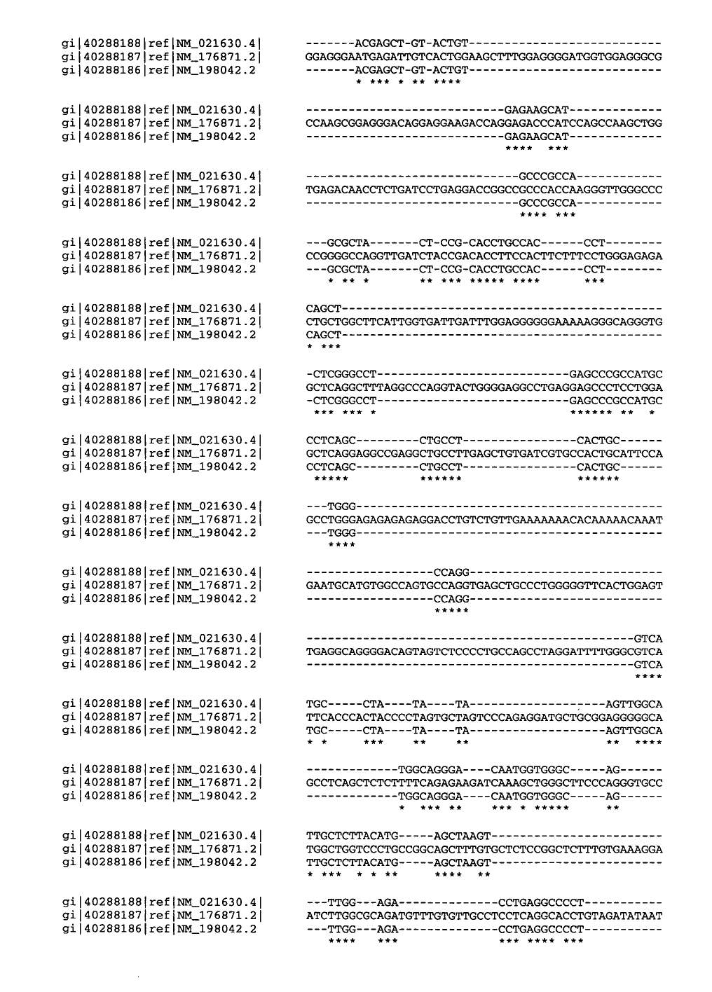

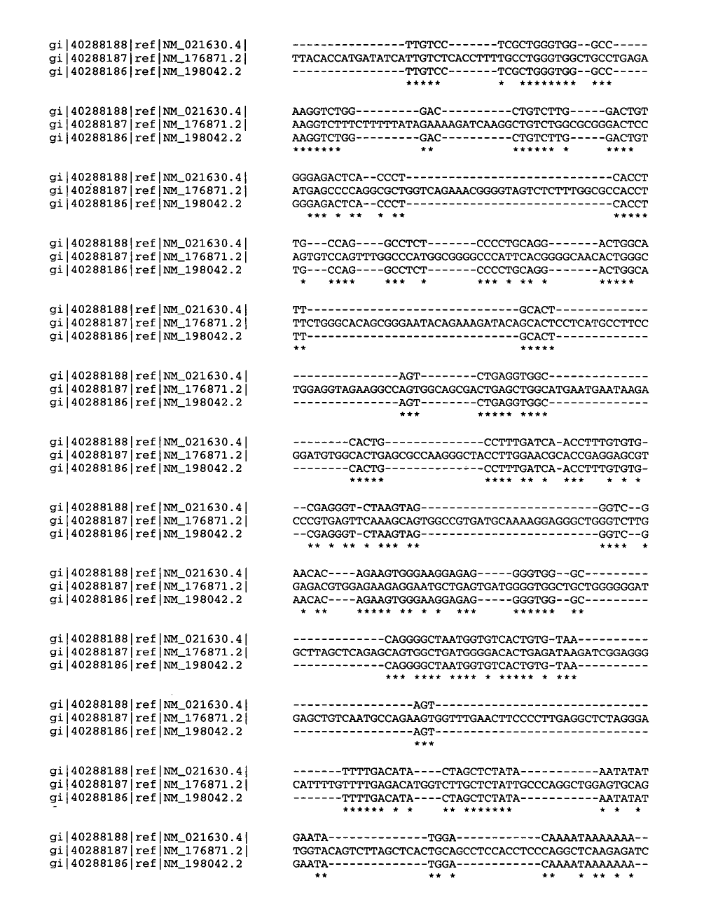

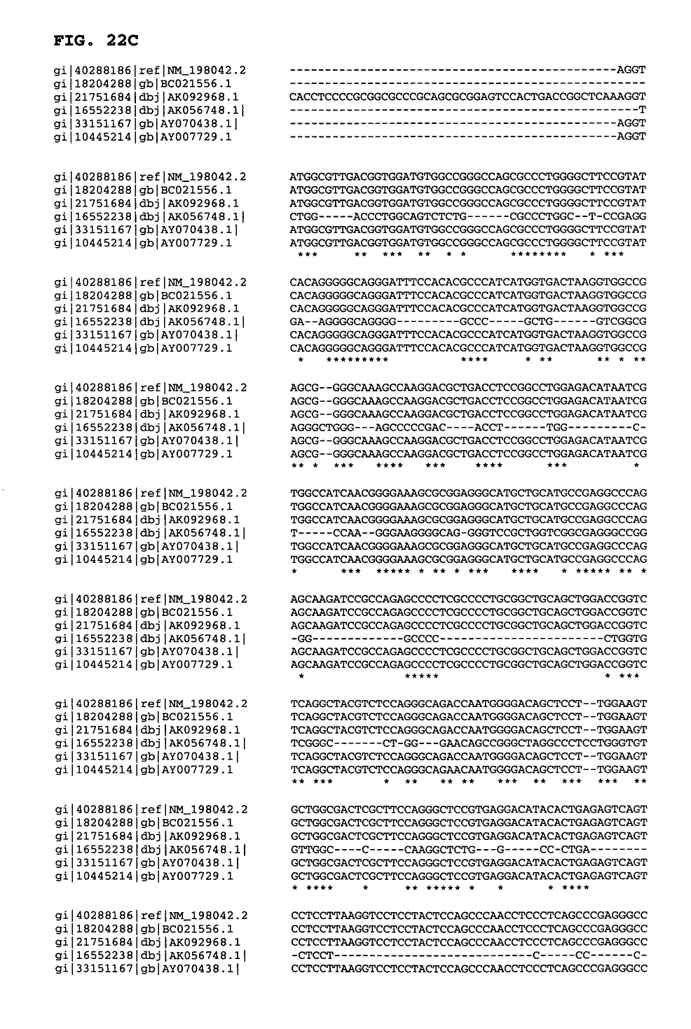

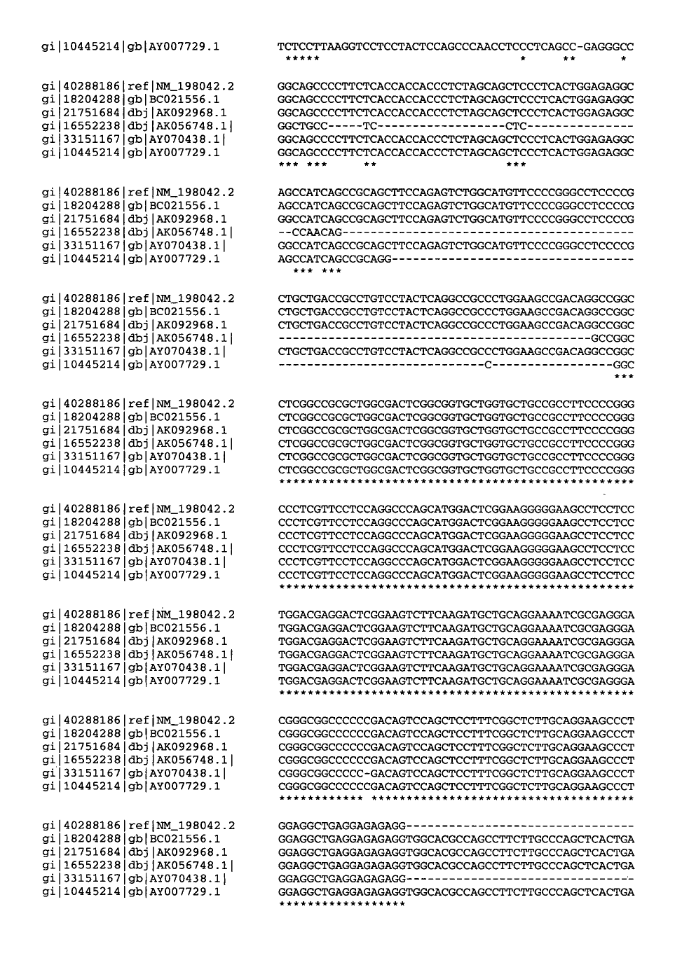

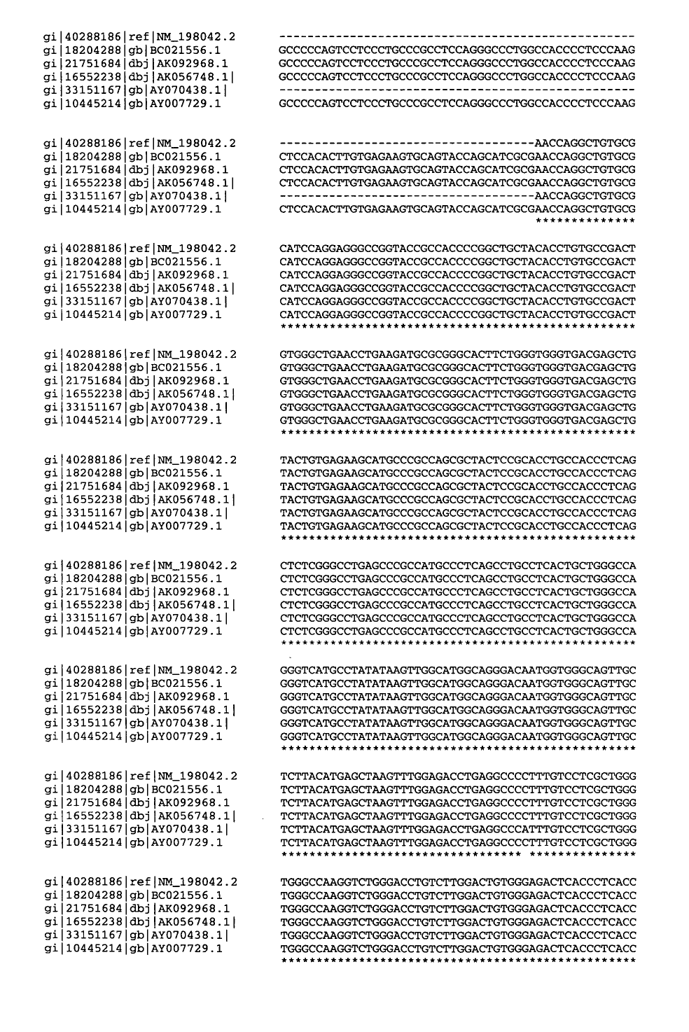

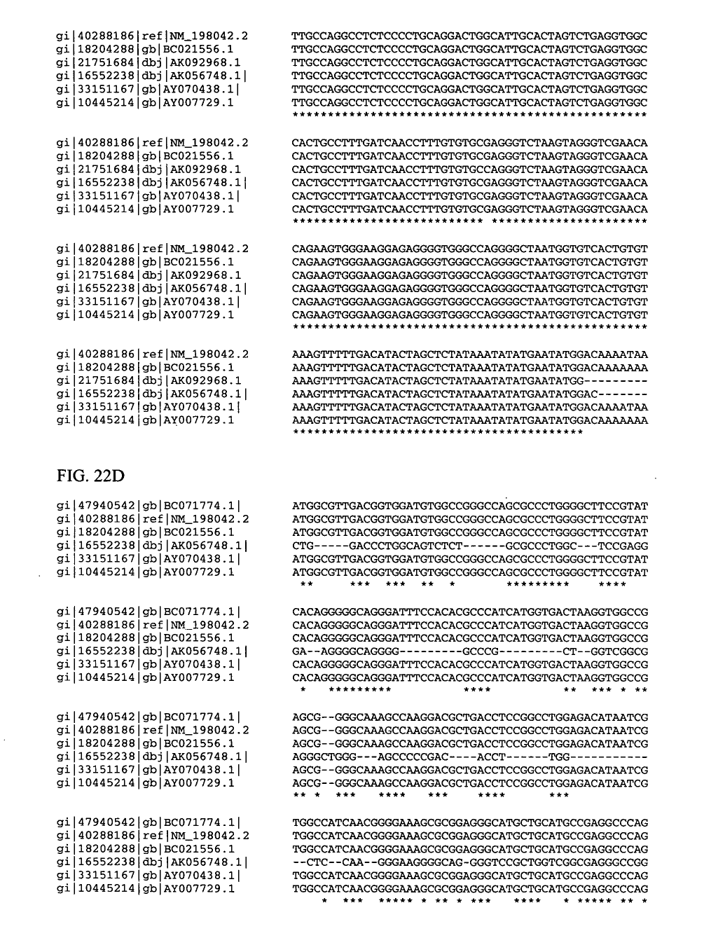

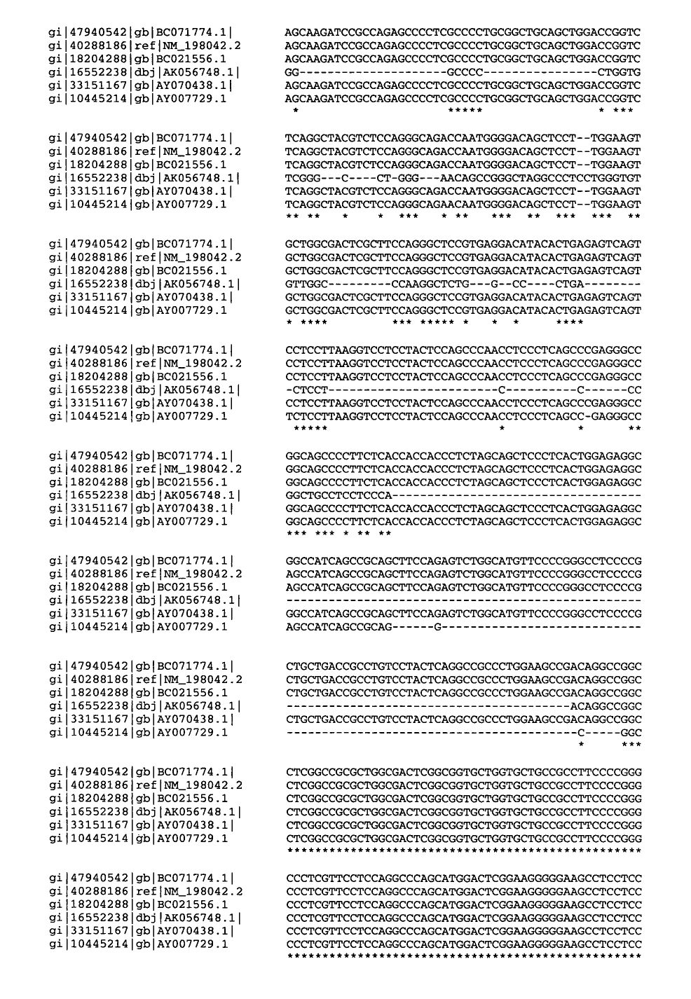

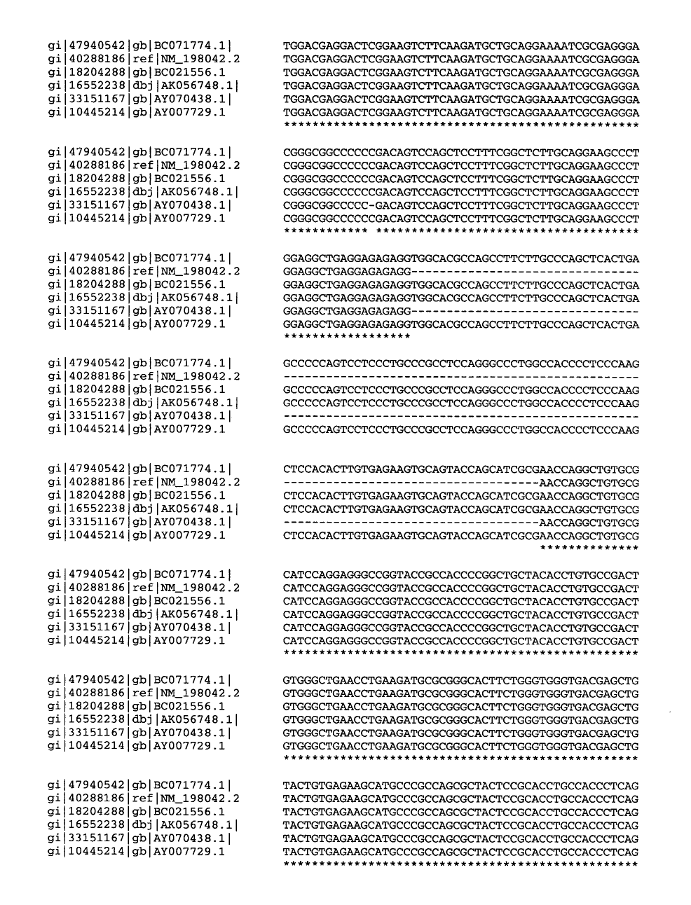

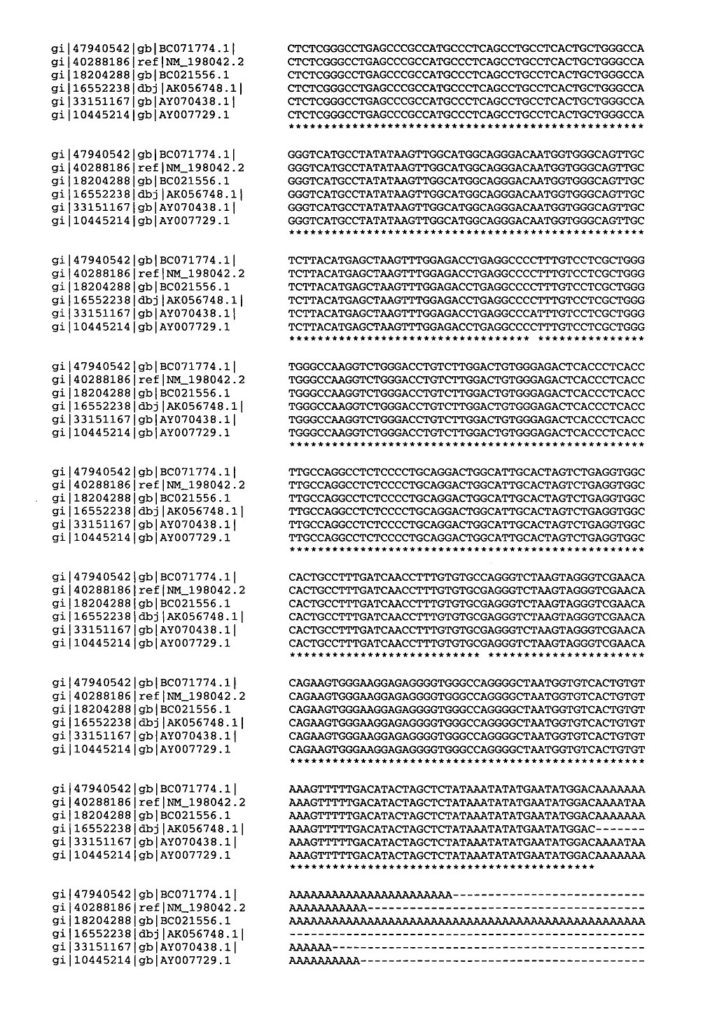

Based on bioinformatic analysis, hSLIM protein carries a PDZ motif at its N-terminus and a LIM domain at its C-terminus (FIG. 1). The LIM domain contains a conserved cysteine-rich domain of 40 to 60 residues, known as C3HC4 zinc-finger or RING finger, found that binds two atoms of zinc. The 3D structure of the zinc-binding motif is referred to as a “cross-brace” motif and seems to be critical for the enzymatic activity of Ring finger proteins (Lorick et al., Proc. Natl. Acad. Sci. U.S.A. 96, 11364, 1999). Based on sequence alignment analysis comparing the RING domain found in hSLIM with similar domains from enzymatically active proteins, we determined the location of 2 critical cysteines at amino acids 286 and 289 (see FIG. 13). Using the QUICKCHANGE MUTAGENESIS™ kit (Stratagene), C286 and C289 were mutated to serines, generating Mu-hSLIM, aka Mu-SLIM C1C2. The Mu-hSLIM sequence containing these 2 mutations was cloned into various expression systems, allowing the expression of bacterial GST-Mu-SLIM, aka GST-Mu-SLIM C1C2 and mammalian Mu-SLIM.

### EXAMPLE 4

### Production of Recombinant Human-SLIM Protein

Bacterially expressed GST-SLIM: The open reading frame encoding hSLIM cDNA was cloned into the bacterial expression plasmid pGEX6 (Amersham) using EcoR1 and Not1 restriction sites. GST-hSLIM was expressed in E. coli BL-21 induced with IPTG. Cells were lysed by sonication in Tris-Cl buffer, pH 8.0 containing 0.5% TRITON® X-100, lysosyme, and protease inhibitors. -Inclusion bodies were resuspended in Tris-Cl buffer pH 8.0 containing 50 mM glycine and 8.5M urea, and dialyzed against Tris-Cl pH 8.0 to allow protein refolding. Protein extracts were purified using glutathione resin (Amersham) and eluted with 5 mM glutathione.

Baculoviral expressed hemagluttinin-tagged SLIM (HA-SLIM): The open reading frame encoding hSLIM cDNA was cloned into the baculoviral expression plasmid pBluBac 4.5 (Invitrogen), which contains a polyhedrin promoter and supports strong protein expression. Virus was generated by co-transfection of pBlueBac-hSLIM and Bac-N-Blue vectors into Sf9 insect cells. Protein was generated in HIGH-FIVE™ cells transduced with hSLIM encoding virus. Cells were lysed by sonication in 1% TRITON® X-100 buffer containing protease inhibitors. Recombinant hSLIM protein was purified by affinity purification using anti-HA matrix (Roche).

HA-SLIM expressed in Mammalian cells: The open reading frame encoding hSLIM cDNA was cloned into the mammalian expression plasmid pCDNA3.1 (Invitrogen), which contains a CMV promoter and supports strong protein expression. The HA tag was inserted either at the amino- or carboxy- terminus of the SLIM protein sequence. Recombinant hSLIM protein was generated by transient transfection of 293T cells. Cells were lysed by sonication in 1% TRITON® X-100 buffer containing protease inhibitors, and recombinant hSLIM protein was purified by affinity purification using anti-HA matrix (Roche).

### EXAMPLE 5

### Ubiquitin Transfer Assay

SLIM ubiguitination reactions: Recombinant human SLIM protein was incubated in the presence of ATP, rabbit E1, human recombinant E2, and biotinylated ubiquitin (Boston Biochem) in a reaction buffer containing 1 mM DTT and 5 mM MgCl₂. The reaction took place at 30° C., for 3-16 hours in 20 μl total volume.

STAT ubiguitination by SLIM: recombinant human STAT1 (Biosource) or recombinant STAT4 (Abnova) were included in the reaction described above, and incubated at 30° C. for 3-16 hours. Samples were separated on SDS-PAGE, transferred to PVDF, and immunoblotted with Avidin-HRP (Becton Dickinson). Blots were overlayed with anti-SLIM antibody or anti-STAT antibody.

### EXAMPLE 6

### In vitro Knock Down by RNAi Transfection

Human primary CD4+ T cells were isolated from buffy coat by negative selection using MACS® T cell isolation kit (Miltenyi). The human Jurkat cell line and primary human CD4+ T cells were transfected with siRNA by electroporation, using a T cell line NUCLEOFECTOR™ kit (Amaxa). The sequence of the siRNA oligonucleotides used to effectively knock down (KS) endogenous hSLIM expression were:

| | | |
|---|---|---|
| SL1: | ACA TAA TCG TGG CCA TCA A | (SEQ ID NO: 15) |
| SL2: | GAG AGT CAG TCC TCC TTA A | (SEQ ID NO: 16) |

Gene-specific knock down (KD) was verified by western blotting with anti-SLIM pAb in Jurkat cells and by RT-PCR in CD4+ T cells. Off target effect was verified by western blotting for various proteins, including actin and STAT1.

### EXAMPLE 7

### Cytokine Production in Human CD4+ T Cells

In order to determine the effect of SLIM knock down on T cell biological responses, we investigated the effect of in vitro knock-down (KD) on human interferon gamma (IFNγ) production. Untransfected and RNAi-transfected cells were either left untreated or stimulated with anti-CD3+/anti-CD28 in the presence or absence of IFNα or IL12 (R and D Systems). Production of human IFNγ was investigated in individual cells by FACS analysis and in cell culture supernatants by indirect sandwich ELISA.

For intracellular FACS, cells were treated with Brefeldin A for 3 hrs prior to immunostaining. Cells were stained CD69 surface expression, washed and then permeabilized using CYTOFIX/CYTOPERM™ (BD Biosciences). Permeabilized cells are stained with control or anti-IFNγ antibody coupled to phycoerythrin (PE).

Human IFNγ ELISA was performed using Nunc MAXISORP™ 96-well plates coated with a mouse anti-human IFNγ monoclonal antibody (BioSource) in a carbonate/bicarbonate coating buffer, and blocked with SUPERBLOCK® Blocking Buffer (Pierce). The presence of IFNγ in cell culture supernatants were detected using a biotinylated mouse anti-IFNγ monoclonal antibody (BioSource), in a SA-HRP secondary (BD Biosciences) and TMB substrate system (KPL).

### EXAMPLE 8

### Effect of SLIM on STAT1 and STAT4 Transcriptional Activity

A total of 2×10⁵ 293T cells were transiently transfected with plasmid cDNA for GAS-Luc alone, STAT1+ GAS-Luc, or SLIM+ STAT1+ GAS-Luc. In some experiments, STAT4 was substituted for STAT1. Renilla luciferase was included in all transfections as a transfection efficiency control. Duplicate samples were grown for 36-48 hrs. Cells were either left unstimulated or treated with a 500U IFNα or 50 ng/ml IFNγ for 16 hrs. The cells were washed and lysed in luciferase buffer (Promega), and luciferase activity was measured using 20-50 μg of protein.

### EXAMPLE 9

### Association of SLIM and STAT proteins

In order to determine whether human SLIM associates with STAT1 and/or STAT4 in vivo, 293T cells were transiently transfected with in the following combinations of cDNA: STAT1 alone, SLIM alone or SLIM+STAT1. Duplicate samples were grown for 48 hr. Cells were either left untreated or were treat with 50ng/ml IFNγ for 15-20 min. Cells were lysed in 0.5% TRITON® X-100 buffer with protease and phosphatase inhibitors, and immunoprecipitated using anti-STAT1 Ab (Santa Cruz). Western blots were probed with anti-SLIM pAb followed by anti-STATl overlays. In some experiments, STAT4 cDNA was substituted for STAT1. In this case, cells were either left untreated or were treat with 500U/ml IFNα for 15-20 min and immunoprecipitated using anti-STAT4 pAb (Santa Cruz).

### EXAMPLE 10

### Generation of Antibodies Specific to Human SLIM Protein

Human SLIM shows homology at its amino- and carboxy termini with other proteins that carry N-terminal PDZ domain and C-terminus LIM domain. However, the amino acid sequence outside these 2 domains (i.e., the center portion of SLIM) are unique to human SLIM. In order to generate antibodies specific to human SLIM and minimize cross reactivity to PDZ and LIM proteins, we generated a vector expressing human SLIM lacking the PDZ and LIM domains, i.e., ΔPDZ-ΔLIM-SLIM or the middle portion of human SLIM. The ΔPDZ-ΔLIM-SLIM was PCR amplified and cloned into pET21b(+) (Novagen) between restrictions sites EcoRI (5′) and XhoI (3′). The Histidine tag was at the C-terminus of the ΔPDZ-ΔLIM-SLIM. The start codon was at the T7 tag.

EcoRI-site primer for ΔPDZ-ΔLIM-SLIM (no PDZ/LIM):

(SEQ ID NO: 17)

TTAAGAATTCAGGGCAGACCAATGGGGACAGCTCCTTGGAAGTGC

XhoI-site primer for ΔPDZ-ΔLIM-SLIM (no PDZ/LIM):

(SEQ ID NO: 18)

TTAACTCGAGAGGGGTGGCCAGGGCCCTGGAGGCGGGCAGGGAG

Amino acid sequence of C-His-ΔPDZ-ΔLIM-SLIM: (i.e., 200 amino acids, ˜21 kDa):

| | |
|---|---|
| MGRDPNSGQTNGDSSLEVLATRFQGSVRTYTESQSSLRSSYSSPTSLSPRAGSPFSPPPSSSSLTGEAAIS | (SEQ ID NO: 19) |
| RSFQSLACSPGLPAADRLSYSGRPGSRQAGLGRAGDSAVLVLPPSPGPRSSRPSMDSEGGSLLLDEDSEVF | |
| KMLQENREGRAAPRQSSSFRLLQEALEAEERGGTPAFLPSSLSPQSSLPASRALATPLEHHHHHH | |

C-His-ΔPDZ-ΔLIM-SLIM protein was expressed in E. coli BL-21. Cells were lysed by sonication in 100 mM NaH₂PO4 pH 8.0, 10 mM Tris HCl, pH 8.0, 8M urea and then incubated with Nickel-NTA resin (QIAGEN). The resin was washed and protein eluted with 50 mM imidazole. The purified protein was injected with adjuvant into rabbits for polyclonal Ab production.

### EXAMPLE 11

### E3 Ligase Activity of SLIM Isoform WT-Δ52-hSLIM

GST-Δ52-hSLIM protein (corresponding to isoform 3, partial deletion that maintains PDZ and LIM domains, expressed in CD4+ cells) was expressed and purified from E. coli as described in Example 4. The GST-Δ52-hSLIM protein was incubated in the presence of E1, E2 and ubiquitin at 30° C. Proteins were separated on NuPage gels and blotted with Avidin-HRP. The blot was overlayed with anti-SLIM pAb to show SLIM loading.

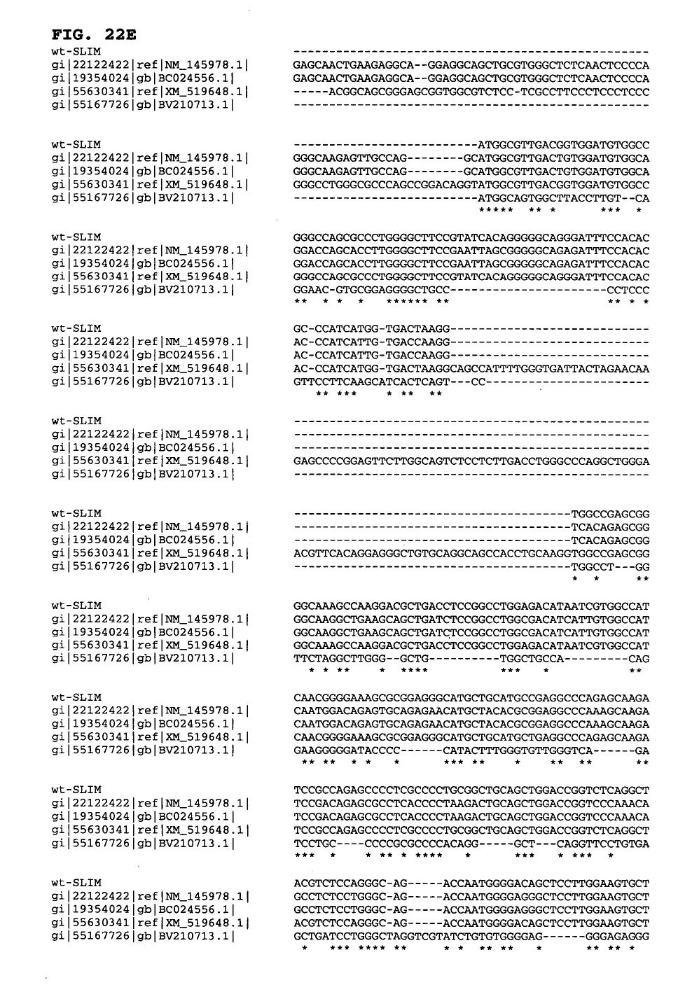

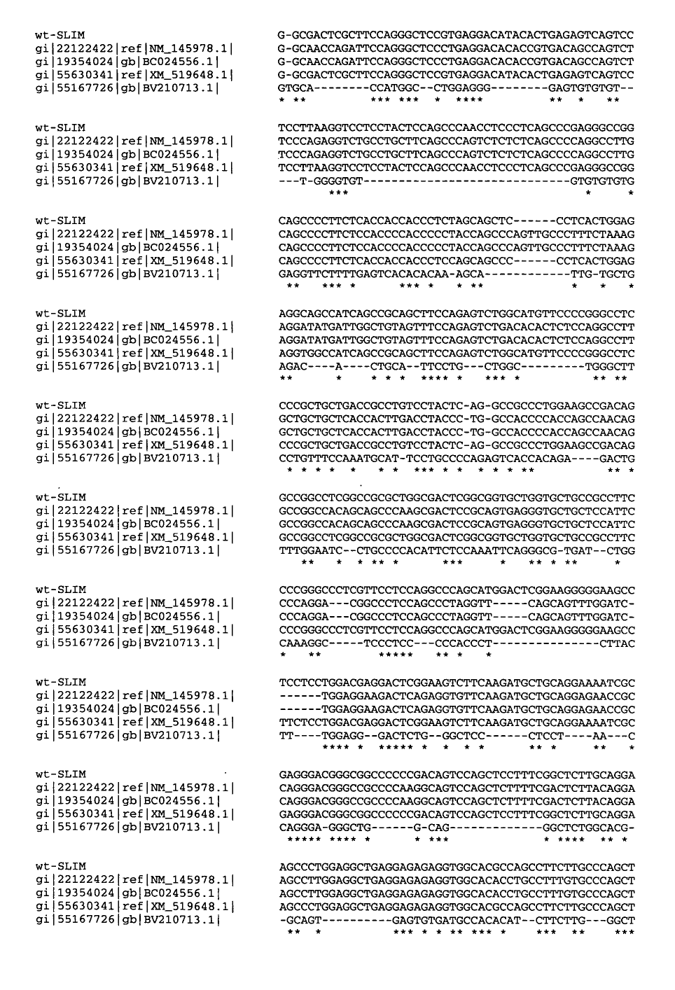

The results, which are shown in FIG. 24, show that WT-Δ52-hSLIM demonstrates E3 ligase activity by inducing its own ubiquitination. Ubiquitin transfer occurs in a dose-dependent manner. WT-Δ52-hSLIM exhibits enhanced activity compared to WT-hSLIM.

### EXAMPLE 12

### Ubiquitination of STAT-1 by WT-SLIM and WT-Δ52-hSLIM

GST-Δ52-hSLIM protein (corresponding to isoform 3, partial deletion that maintains PDZ and LIM domains, expressed in CD4+ cells) was incubated in the presence of E1, E2, recombinant STAT1, and ubiquitin at 30° C. Proteins were separated on NuPage gels and blotted with Avidin-HRP. The blot was overlayed with anti-STAT1 pAb to show loading.

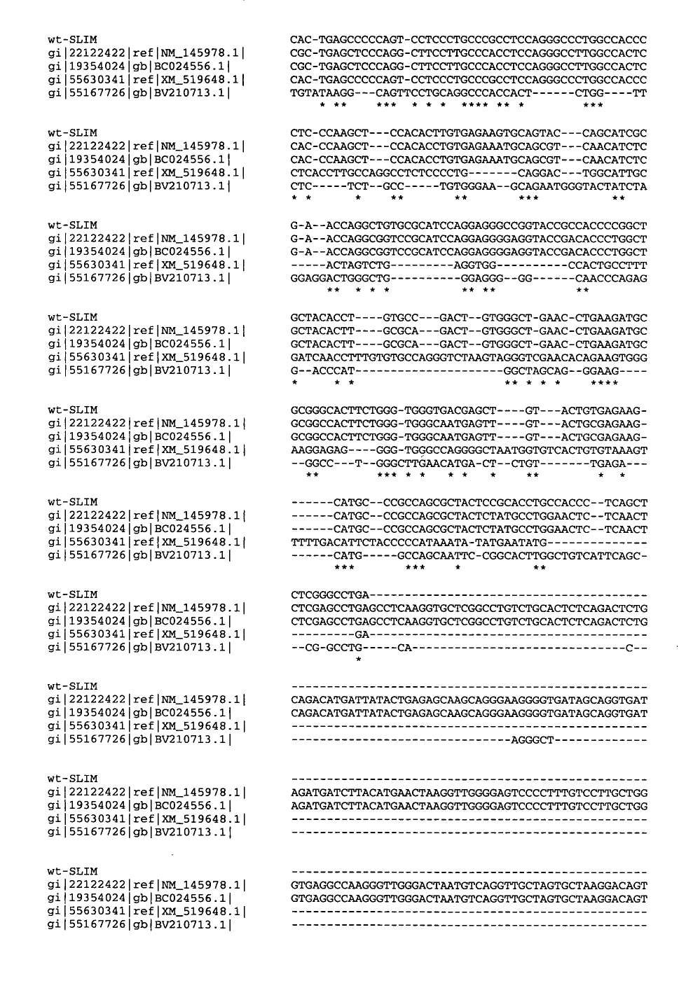

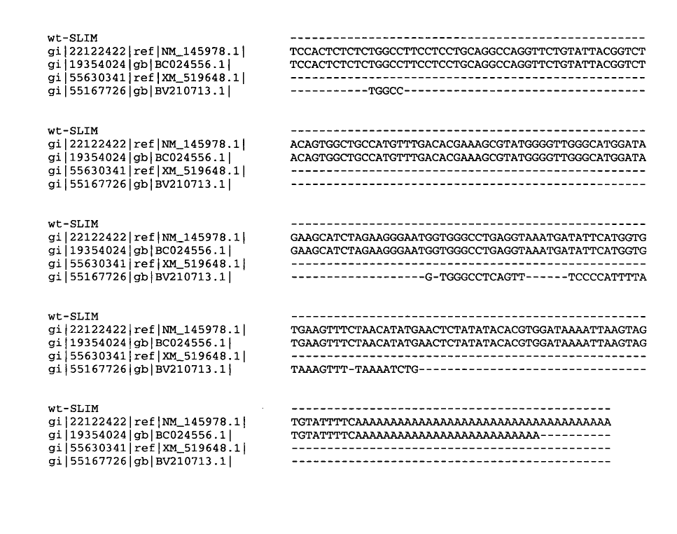

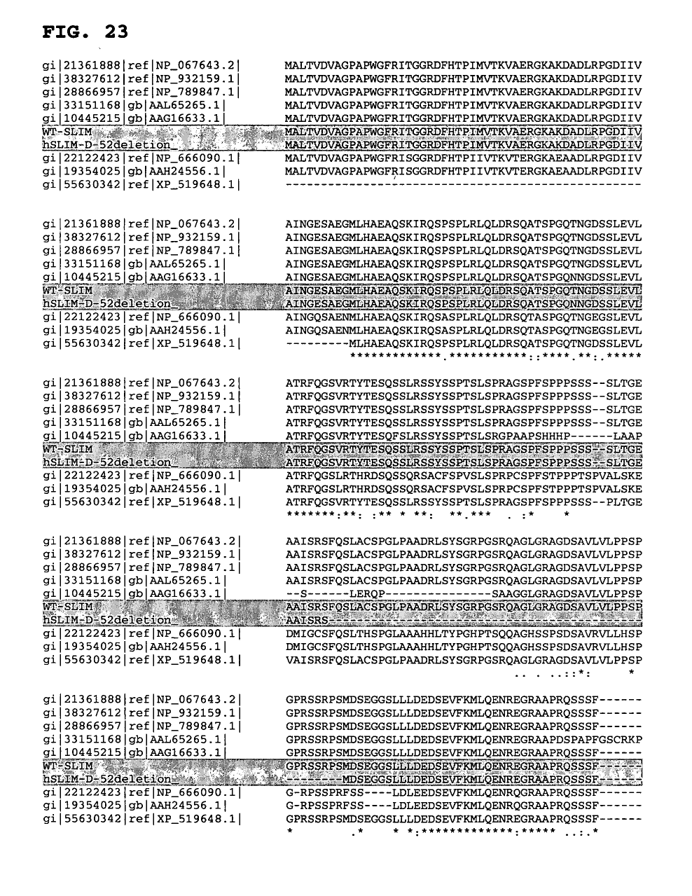

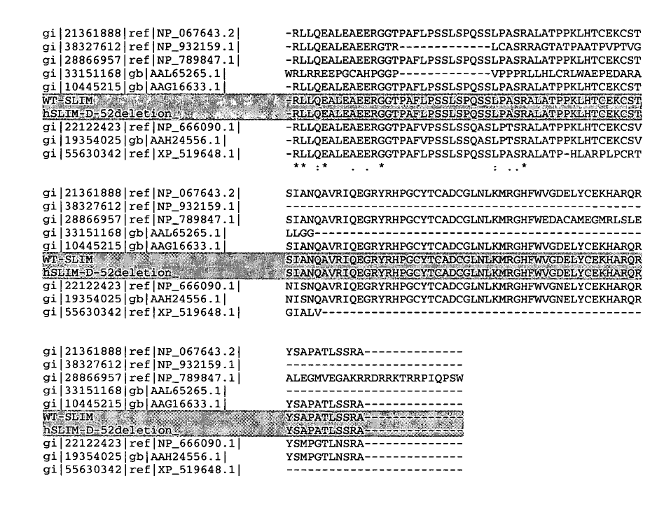

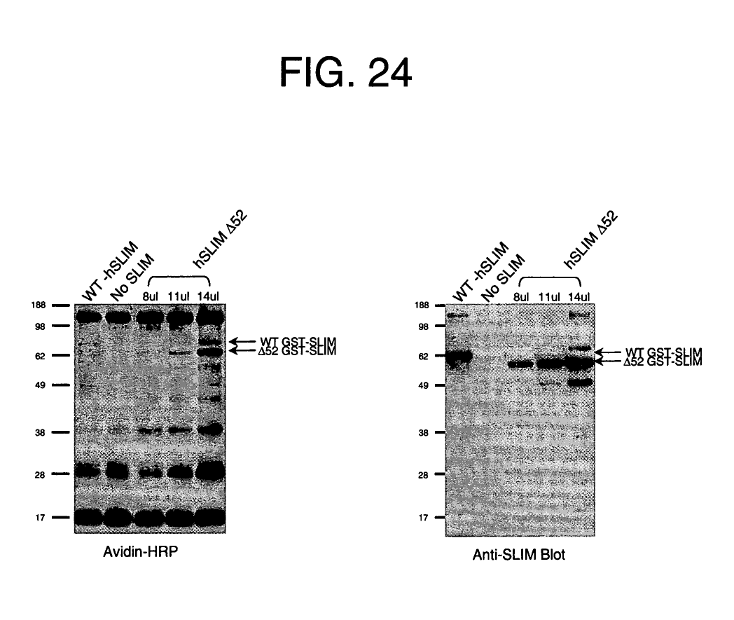

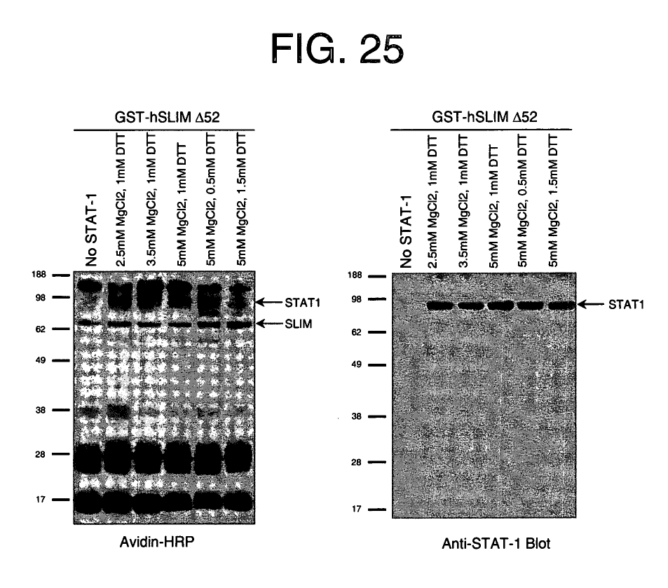

The results, which are shown in FIG. 25, show that WT-Δ52-hSLIM demonstrates E3 ligase activity by transferring ubiquitin to a biologically relevant downstream substrate, STAT1.

### EXAMPLE 13

### Identification of Human E2 Enzymes that Facilitate WT-Δ52-hSLIM Ubiquitination

Ubiquitin transfer occurs via a cascade of 3 enzymes, E1, E2 and E3. A certain degree of specificity is imparted by E2, while most of the substrate specificity is conveyed by E3. In this assay, we determined which of the most likely 8 human E2 enzymes preferentially mediates ubiquitin transfer by SLIM.

GST-A52-hSLIM protein was incubated in the presence of E1, two human E2 isoforms and ubiquitin at 30° C. Proteins were separated on NuPage gels and blotted with Avidin-HRP. Additional E2 enzymes were evaluated, including UbcH2, H3, H5b, H5c, H6, H7 and H10.

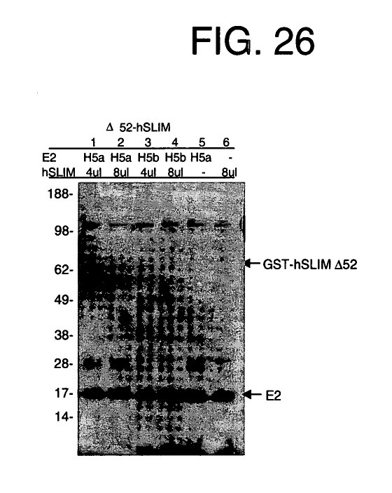

The results, which are shown in FIG. 26, show that WT-A52-hSLIM demonstrates preferential ubiquitination in the presence of UbcH5a.

### EXAMPLE 14

### Generation of LIM-Domain Point Mutants of hSLIM

Alignment of hSLIM LIM domain with Ring motifs from other proteins revealed the presence of conserved cysteines at positions corresponding to C1, C2, C5 and C6 of the consensus site. The first 2 cysteines are located in loop 1 and the second 2 cysteines in loop 2 of the LIM domain.

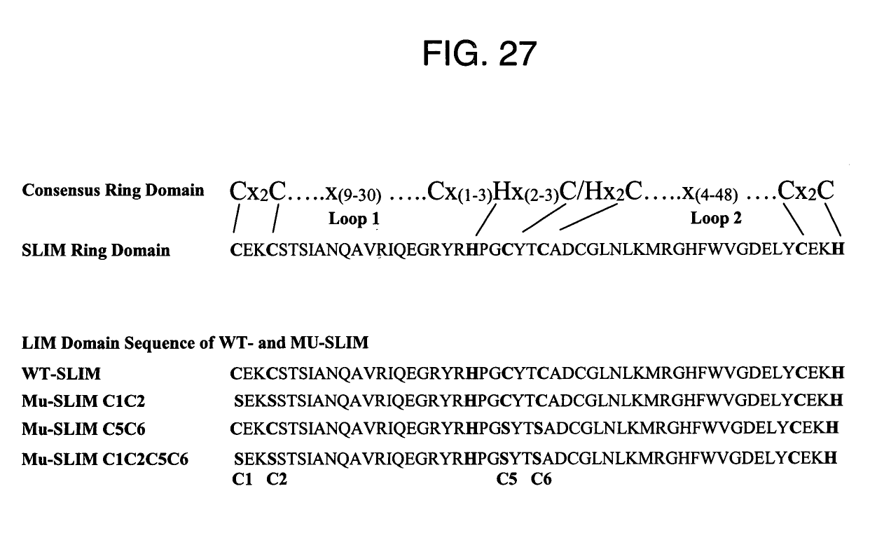

We generated three LIM domain point mutants. The sequences of these mutants are shown in FIG. 27. Mu-SLIM C1C2 carries cysteine to serine substitutions at C286 and C289. Mu-SLIM C5C6 carries cysteine to serine substitutions at C310 and C312. All 4 cysteines were mutated to serines in Mu-SLIM C1C2 C5C6.

### EXAMPLE 15

### Expression and Purification of WT- and Mu- GST-hSLIM

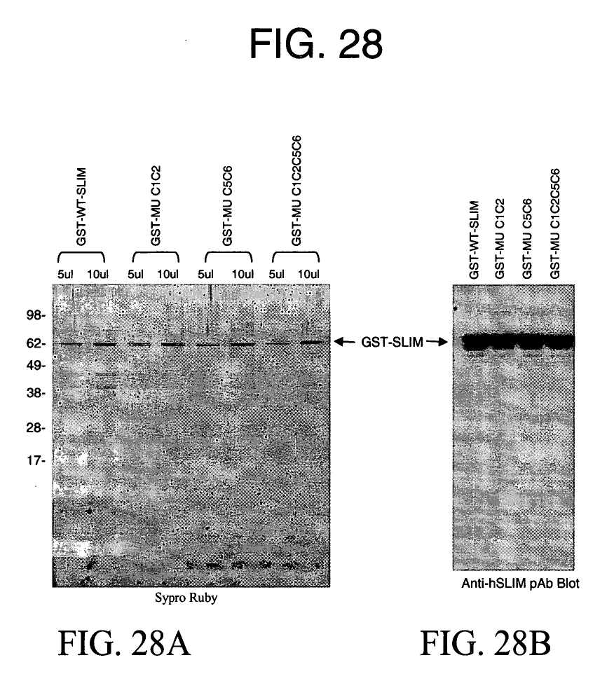

WT- and Mu-hSLIM genes were cloned in a bacterial expression system. Protein was expressed and purified as descried above. The sypro ruby stained gel (FIG. 28A) and the anti-SLIM western blot (FIG. 28B) demonstrate expression and purification of the recombinant hSLIM proteins.

### EXAMPLE 16

### Ubiquitin Transfer Activity of WT and Mu-hSLIM

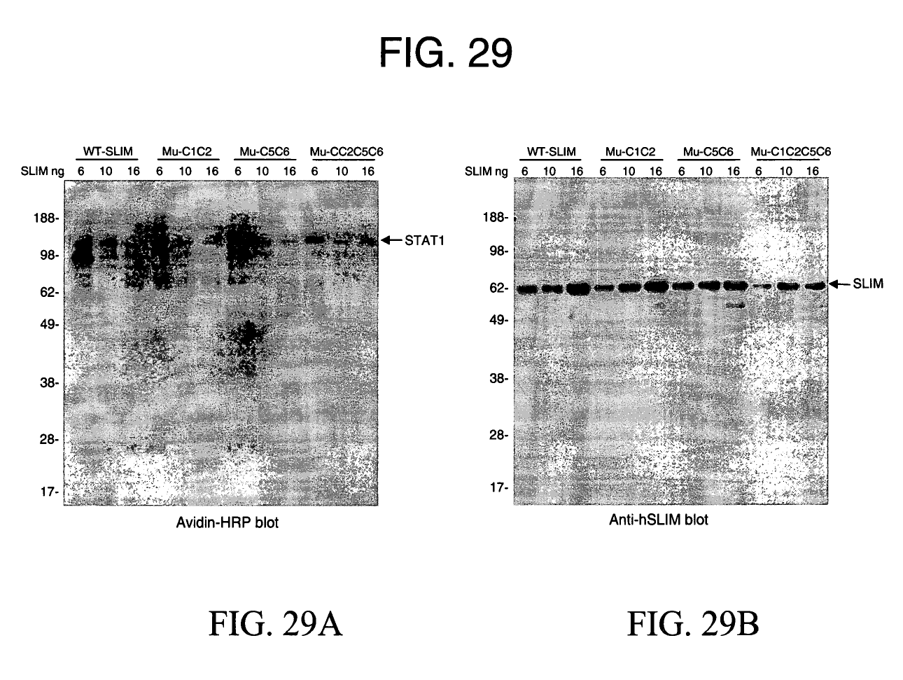

WT- and Mu-GST-SLIM proteins were incubated in the presence of E1, E2, ubiquitin and human STAT1. Proteins were separated on NuPage gels and blotted with Avidin-HRP (FIG. 29A). The blot was overlayed with anti-STAT1mAb to show SLIM loading (FIG. 29B).

Comparing the ubiquitin transfer activity of WT and LIM domain point mutants of hSLIM showed that point mutations of conserved cysteines in either loop 1 or loop 2 of the LIM domain reduces the ubiquitin transfer activity of SLIM and prevents the ubiquitination of the STAT substrate.

### EXAMPLE 17

### Ubiquitin Transfer Activity of Recombinant GST-hSLIM

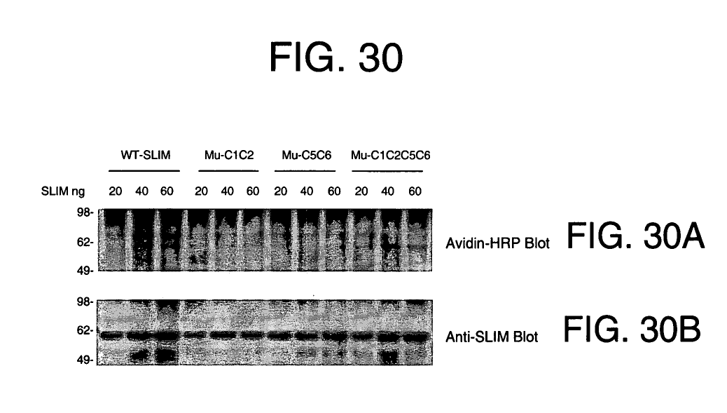

WT- and Mu-GST-SLIM proteins were incubated in the presence of E1, E2, and ubiquitin to compare the ubiquitin transfer activity of wild type and point mutants of hSLIM. Proteins were separated on NuPage gels and blotted with Avidin-HRP (FIG. 30A). The blot was overlayed with anti-SLIM pAb to show SLIM loading (FIG. 30B).

Point mutations of conserved cysteines in the LIM domain of hSLIM inhibits ubiquitin transfer onto the SLIM protein and reduces its ubiquitin transfer activity.

### EXAMPLE 18

### Effect of LIM-Domain Point Mutants on STAT1 Activity

293T cells were transiently transfected with either wild type (WT)- or point mutant (Mu)-SLIM, along with STAT1 and a reporter construct encoding the GAS-response element. Cells were either left untreated, or stimulated with IFNγ, and analyzed for GAS-Luc activity.

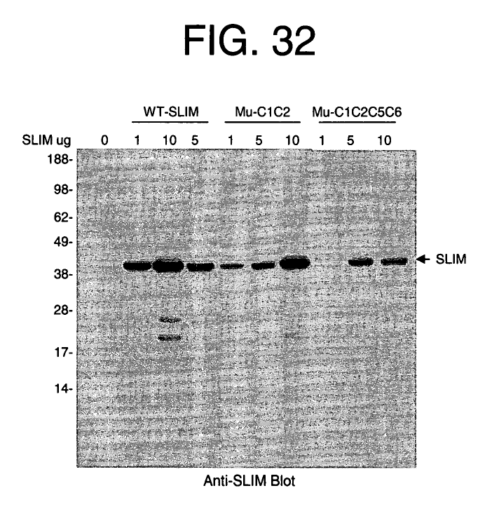

To control for the STAT1 transcriptional assay, 293T cells were transiently transfected with either wild type (WT)- or point mutant (Mu)-SLIM, along with STAT1 and a reporter constructs encoding the GAS-response element. Cells were lysed, and analyzed for SLIM expression. WT- and point mutants of hSLIM are expressed in a dose dependent manner in 293T cells. The results are shown in FIG. 32.

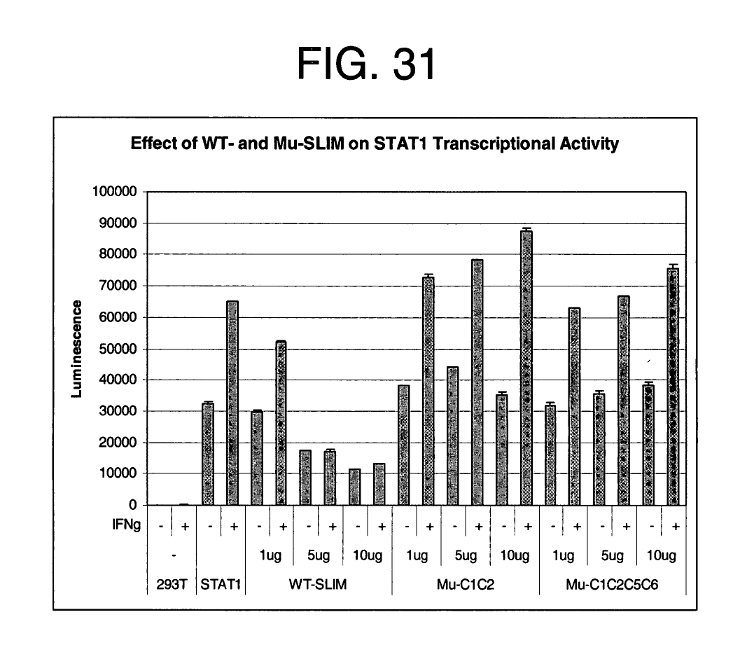

The results are shown in FIG. 31. Treatment with IFNγ enhances STAT1-mediated GAS-luc activity, which is inhibited by WT-SLIM in a dose dependent manner. In contrast, both hSLIM mutants had little effect on GAS-Luc activity.

### EXAMPLE 19

### Association of UbcH5 and UbcH6 with hSLIM

Jurkat E6.1 cells were lysed in Triton (T), octyl β Glucoside (OβG) or Brij-35 (B). Cell lysates were cleared by centrifugation and were incubated with WT-hSLIM bound to sepharose beads. The samples were separated on NuPage gels and analyzed by Western blotting for UbcH3, H5, H6, H7, and H10 binding. Whole cell lysates were used as Western blotting controls (E6.1 wcl).

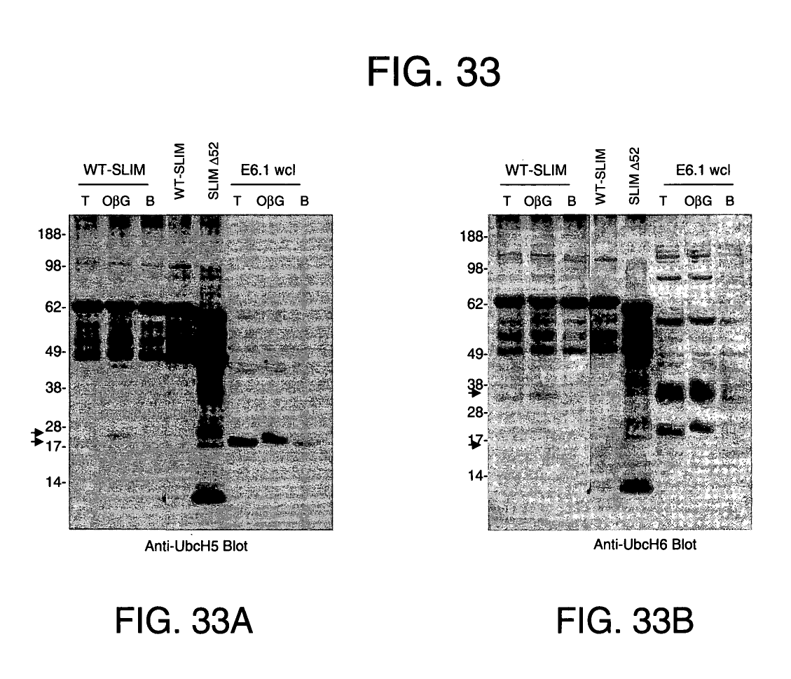

FIG. 33 demonstrates that WT-hSLIM associates preferentially with 2 of the 5 ubiquitin-conjugating enzymes tested (UbcH6 and UbcH6).

1. An isolated and purified polypeptide which
i. comprises the amino acid sequence shown in SEQ ID NO:4;
ii. comprises the amino acid sequence shown in SEQ ID NO:49; or
iii. consists of the amino acid sequence shown in SEQ ID NO: 19.

2. An isolated and purified nucleic acid molecule comprising:
iv. a coding sequence for the polypeptide of claim 1; or
v. the complement of the coding sequence,
wherein the coding sequence does not encode SEQ ID NOS:2, 3, 5-9, or 41-45.

3. The nucleic acid molecule of claim 2 which is:
vi. double-stranded;
vii. single-stranded;
viii. RNA;
ix. cDNA;
x. a vector; or
xi. an expression construct comprising the coding sequence and a promoter located upstream from the coding sequence which controls expression of the coding sequence;

4. A method of producing the polypeptide of claim 1, comprising:
(i) culturing a host cell comprising the nucleic acid molecule of claim 2, wherein the nucleic acid molecule is the expression construct, in a culture medium under conditions whereby the host cell expresses the polypeptide; and
(ii) recovering the polypeptide from the culture medium or a host cell lysate.

5. The method of claim 4 further comprising purifying and/or refolding the polypeptide in the presence of zinc or in the absence of EDTA, DTPA, TPEN, and EGTA.

6. A method of producing enzymatically active wild-type hSLIM comprising the amino acid sequence shown in SEQ ID NO:2, comprising:
(a) purifying the wild-type hSLIM; and
(b) refolding the wild-type hSLIM in the presence of zinc and, optionally, purifying and/or refolding the wild-type hSLIM in the absence of EDTA, DTPA, TPEN, and EGTA.

7. An antibody which specifically binds hSLIM or a portion thereof which is not a PDZ or a LIM domain and optionally comprises a detectable label.

8. The antibody of claim 7 which is a polyclonal antibody, a monoclonal antibody, a chimeric antibody, a humanized antibody, a human antibody, an Fab fragment, an F(ab′)₂ fragment, an Fv fragment, a single-chain antibody, or an intracellular antibody.

9. A method for identifying compounds which interfere with binding of an hSLIM polypeptide to a STAT protein, comprising:
(a) contacting a first polypeptide, a second polypeptide, and a test compound, wherein either:
(1) the first polypeptide comprises an hSLIM polypeptide and the second polypeptide comprises a STAT protein; or
(2) the first polypeptide comprises the STAT protein and the second polypeptide comprises the hSLIM polypeptide; and
(b) determining the quantity of the first polypeptide which is bound to, is displaced from, or is prevented from binding to, the second polypeptide; and
(c) identifying the test compound is identified as an agent which interferes with the binding of the hSLIM polypeptide to the STAT protein if the test compound:
(1) diminishes the quantity of the first polypeptide bound to the second polypeptide;
(2) displaces first polypeptide bound to the second polypeptide; or
(3) prevents first polypeptide from binding to the second polypeptide.

10. A method for identifying compounds which interfere with STAT1- or STAT4-mediated transcription, comprising:
(a) contacting a test compound, a first polypeptide, a second polypeptide, and a reporter construct comprising a STAT1- or STAT4-binding sequence upstream from a reporter gene to form a transcription mixture, wherein either:
(1) the first polypeptide comprises an hSLIM polypeptide and the second polypeptide comprises STAT1 or STAT4; or
(2) the first polypeptide comprises STAT1 or STAT4 and the second polypeptide comprises the hSLIM polypeptide;
(b) contacting the transcription mixture with IFNα if the transcription mixture comprises STAT4 or contacting the transcription mixture with IFNγ if the transcription mixture comprises STAT1;
(b) assaying for expression of the reporter gene; and
(c) identifying the test compound as an agent which interferes with STAT1- or STAT4-mediated transcription if expression of the reporter gene in the presence of the test compound is less than expression of the reporter gene in the absence of the test compound.

11. A method for identifying compounds which interfere with binding of hSLIM to STAT1 or STAT4, comprising:
(a) contacting a test compound and a cell which comprises three recombinant DNA constructs, wherein:
(1) a first construct encodes a first polypeptide fused to a sequence-specific DNA-binding domain;
(2) a second construct encodes a second polypeptide fused to a transcriptional activation domain; and
(3) a third construct comprises a reporter gene downstream from a DNA element which is recognized by the sequence-specific DNA-binding domain, and wherein either:
(i) the first polypeptide comprises an hSLIM polypeptide and the second polypeptide comprises STAT1 or STAT4; or
(ii) the first polypeptide comprises STAT1 or STAT4 and the second polypeptide comprises the hSLIM1 polypeptide;
(b) contacting the cell with a test compound;
(c) determining expression of the reporter gene in the presence of the test compound; and
(d) identifying the test compound as an agent which interferes with the binding of hSLIM to STAT1 or STAT4 if the expression of the reporter gene in the presence of the test compound is less than expression of the reporter gene in the absence of the test compound.

12. A cell which comprises three recombinant DNA constructs, wherein:
(a) a first construct encodes a first polypeptide comprising a sequence-specific DNA-binding domain;
(b) a second construct encodes a second polypeptide comprising a transcriptional activation domain; and
(c) a third construct comprises a reporter gene downstream from a DNA element which is recognized by the sequence-specific DNA-binding domain, wherein either
(1) the first polypeptide comprises an hSLIM polypeptide and the second polypeptide comprises a STAT protein; or
(2) the first polypeptide comprises the STAT protein and the second polypeptide comprises the hSLIM polypeptide.

13. A method of identifying agonists or antagonists of E3 ligase activity of hSLIM, comprising steps of:
(a) contacting an hSLIM polypeptide with a test compound; and
(b) assaying the E3 ligase activity of the hSLIM polypeptide, wherein:
(1) the test compound is identified as an agonist of hSLIM E3 ligase activity if the test compound increases E3 ligase activity of the hSLIM polypeptide relative to E3 ligase activity of the hSLIM polypeptide in the absence of the test compound; or
(2) the test compound is identified as an antagonist of hSLIM E3 ligase activity if the test compound decreases E3 ligase activity of the hSLIM polypeptide relative to E3 ligase activity of the hSLIM polypeptide in the absence of the test compound.

14. A composition, comprising:
(a) an active agent selected from the group consisting of:
(1) an hSLIM polypeptide;
(2) a nucleic acid molecule encoding the hSLIM polypeptide;
(3) an siRNA molecule which silences transcription of an hSLIM gene;
(4) an antisense oligonucleotide which prevents transcription of an hSLIM gene;
(5) an antibody which specifically binds to an hSLIM polypeptide;
(b) an immunomodulatory agent selected from the group consisting of:
(1) a vaccine;
(2) a dendritic cell;
(3) a monoclonal antibody; and
(c) a physiologically acceptable vehicle.

15. The composition of claim 14 wherein the active agent is an siRNA molecule and the siRNA molecule is selected from the group consisting of SEQ ID NOS: 15 and 16.

16. A method of altering IFNγ production by a T cell, comprising contacting the T cell with:
(a) an hSLIM polypeptide or (b) a nucleic acid molecule encoding the hSLIM polypeptide, whereby IFNγ production by the T cell is decreased; or
(c) the antibody of claim 7, (d) an siRNA molecule optionally selected from the group consisting of SEQ ID NOS:9 and 10, or (e) (c) an antisense oligonucleotide which hybridizes to a portion of SEQ ID NO:1 or SEQ ID NO:27, whereby IFNγ production by the T cell is increased.


SEQUENCE LISTING (53 sequences)

SEQ ID NO: 1 - DNA, 1059 bp, Homo sapiens
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctga

SEQ ID NO: 2 - protein, 352 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWVGDELYCEKHARQRYSAPATLSSRA

SEQ ID NO: 3 - protein, 325 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQNNGDSSLEVLATRFQGSVRTYTESQFSLRSSYSSPTSLSRGPAAPSHHHPLAAPSLERQPSAAGGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWVGDELYCEKHARQRYSAPATLSSRA

SEQ ID NO: 4 - protein, 300 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWVGDELYCEKHARQRYSAPATLSSRA

SEQ ID NO: 5 - protein, 278 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGTRLCASRRAGTATPAATPVPTVG

SEQ ID NO: 6 - protein, 366 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTCEKCSTSIANQAVRIQEGRYRHPGCYTCADCGLNLKMRGHFWEDACAMEGMRLSLEALEGMVEGAKRRDRRKTRRPIQPSW

SEQ ID NO: 7 - protein, 289 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPDSPAPFGSCRKPWRLRREEPGCAHPGGPVPPPRLLHLCRLWAEPEDARALLGG

SEQ ID NO: 8 - protein, 349 aa, Homo sapiens
MALTVDVAGPAPWGFRISGGRDFHTPIIVTKVTERGKAEAADLRPGDIIVAINGQSAENMLHAEAQSKIRQSASPLRLQLDRSQTASPGQTNGEGSLEVLATRFQGSLRTHRDSQSSQRSACFSPVSLSPRPCSPFSTPPPTSPVALSKEDMIGCSFQSLTHSPGLAAAHHLTYPGHPTSQQAGHSSPSDSAVRVLLHSPGRPSSPRFSSLDLEEDSEVFKMLQENRQGRAAPRQSSSFRLLQEALEAEERGGTPAFVPSSLSSQASLPTSRALATPPKLHTCEKCSVNISNQAVRIQEGRYRHPGCYTCADCGLNLKMRGHFWVGNELYCEKHARQRYSMPGTLNSRA

SEQ ID NO: 9 - protein, 236 aa, Homo sapiens
MLHAEAQSKIRQSPSPLRLQLDRSQATSPGQTNGDSSLEVLATRFQGSVRTYTESQSSLRSSYSSPTSLSPRAGSPFSPPPSSSPLTGEVAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPHLARPLPCRTGIALV

SEQ ID NO: 10 - protein, 12 aa, Homo sapiens
SVRGHTLRVSPP

SEQ ID NO: 11 - DNA, 48 bp, Homo sapiens
ttaagaattcgccaccatggcgttgacggtggatgtggccgggccagc

SEQ ID NO: 12 - DNA, 42 bp, Homo sapiens
ttaagcggccgctcaggcccgagagctgagggtggcaggtgc

SEQ ID NO: 13 - DNA, 25 bp, Homo sapiens
ccgtgaggacatacactgagagtca

SEQ ID NO: 14 - DNA, 22 bp, Homo sapiens
cacctctctcctcagcctccag

SEQ ID NO: 15 - DNA, 19 bp, Homo sapiens
acataatcgtggccatcaa

SEQ ID NO: 16 - DNA, 19 bp, Homo sapiens
gagagtcagtcctccttaa

SEQ ID NO: 17 - DNA, 45 bp, Homo sapiens
ttaagaattcagggcagaccaatggggacagctccttggaagtgc

SEQ ID NO: 18 - DNA, 44 bp, Homo sapiens
ttaactcgagaggggtggccagggccctggaggcgggcagggag

SEQ ID NO: 19 - protein, 207 aa, Homo sapiens
MGRDPNSGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPLEHHHHHH

SEQ ID NO: 20 - protein, 51 aa, Homo sapiens
CEKCSTSIANQAVRIQEGRYRHPGCYTCADCGLNLKMRGHFWVGDELYCEK

SEQ ID NO: 21 - protein, 46 aa, Homo sapiens
CIICREEMVTGAKRLPCNHIFHTSCLRSWFQRQQTCPTCRMDVLRA

SEQ ID NO: 22 - protein, 45 aa, Homo sapiens
CAICRNHIMDLCIECQGVCNHAFHFHCISRWLKTRQVCPLDNREW

SEQ ID NO: 23 - protein, 66 aa, Homo sapiens
CLICIASVKRNQAVWSCSGCFCIFHMPCIQKWAKDSQFLVSSVTDDDFGKKDCPWPCPKCRFEYRY

SEQ ID NO: 24 - protein, 7 aa, Homo sapiens
TCEKCST

SEQ ID NO: 25 - protein, 9 aa, Homo sapiens
RHPGCYTCA

SEQ ID NO: 26 - DNA, 903 bp, Homo sapiens
atggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctga

SEQ ID NO: 27 - DNA, 1869 bp, Homo sapiens
agagtcgggtagacggcagcgggagcggtggcgtctccccgccttccctccctcccgggcctgggcgcccagccggacaggggagcggcagccaggtgagcgcgcccacctgcgcctctccgcgcggcccgccctccccggcgccgggctcctctccgcgcccctgtcggcgcggaaccctggcctcgtccgcggcccagctccctggagcctcgcatcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgccagggtctaagtagggtcgaacacagaagtgggaaggagaggggtgggccaggggctaatggtgtcactgtgtaaagtttttgacatactagctctataaatatatgaatatggacaaaaaaaaaaaaaaaaaaaaaaaaaaaaaa

SEQ ID NO: 28 - DNA, 1857 bp, Homo sapiens
agagtcgggtagacggcagcgggagcggtggcgtctccccgccttccctccctcccgggcctgggcgcccagccggacaggtgagcggcagccaggtgagcgcgcccacctgcgcctctccgcgcggcccgccctccccggcgccgggctcctctccgcgcccctgtcggcgcggaaccctggcctcgtccgcggcccagctccctggagcctcgcatcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaataaaaaaaaaaaaa

SEQ ID NO: 29 - DNA, 2450 bp, Homo sapiens
ttacagtgcccccagagccggctaggggcatggacttcacctcctggtcctctcttcctcaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggaggacgcttgtgctatggagggaatgagattgtcactggaagctttggaggggatggtggagggcgccaagcggagggacaggaggaagaccaggagacccatccagccaagctggtgagacaacctctgatcctgaggaccggccgcccaccaagggttgggcccccggggccaggttgatctaccgacaccttccacttctttcctgggagagactgctggcttcattggtgattgatttggaggggggaaaaagggcagggtggctcaggctttaggcccaggtactggggaggcctgaggagccctcctggagctcaggaggccgaggctgccttgagctgtgatcgtgccactgcattccagcctgggagagagagaggacctgtctgttgaaaaaaacacaaaaacaaatgaatgcatgtggccagtgccaggtgagctgccctgggggttcactggagttgaggcaggggacagtagtctcccctgccagcctaggattttgggcgtcattcacccactacccctagtgctagtcccagaggatgctgcggagggggcagcctcagctctcttttcagagaagatcaaagctgggcttcccagggtgcctggctggtccctgccggcagctttgtgctctccggctctttgtgaaaggaatcttggcgcagatgtttgtgttgcctcctcaggcacctgtagatataatttacaccatgatatcattgtctcaccttttgcctgggtggctgcctgagaaaggtctttctttttatagaaaagatcaaggctgtctggcgcgggactccatgagccccaggcgctggtcagaaacggggtagtctctttggcgccacctagtgtccagtttggcccatggcggggcccattcacggggcaacactgggcttctgggcacagcgggaatacagaaagatacagcactcctcatgccttcctggaggtagaaggccagtggcagcgactgagctggcatgaatgaataagaggatgtggcactgagcgccaagggctaccttggaacgcaccgaggagcgtcccgtgagttcaaagcagtggccgtgatgcaaaaggagggctgggtcttggagacgtggagaagaggaatgctgagtgatggggtggctgctggggggatgcttagctcagagcagtggctgatggggacactgagataagatcggaggggagctgtcaatgccagaagtggtttgaacttccccttgaggctctagggacattttgttttgagacatggtcttgctctattgcccaggctggagtgcagtggtacagtcttagctcactgcagcctccacctcccaggctcaagagatcctcccacagcctcccaggtagctgggactacaggcgtg

SEQ ID NO: 30 - DNA, 1473 bp, Homo sapiens
aagcccctgagtagctgctccgggagctgtggtgccctctccttctccctttggctcctctcctgctgccttacagtgcccccagagccggctaggggcatggacttcacctcctggtcctctcttcctcaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaataaaaaaaaaaaaa

SEQ ID NO: 31 - DNA, 1772 bp, Homo sapiens
gccctccccggcgccgggctcctctccgcgcccctgtcggcgcggaaccctggcctcgtccgcggcccagctccctggagcctcgcgtcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcagccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaaaaaaaaaaaaaaaaaaaaaaaaaaaaaaaaaaaaaaaaaaaaaaaaaaaaaaaaaaaaa

SEQ ID NO: 32 - DNA, 2226 bp, Homo sapiens
agacacacccaggcagcccaccctgcccaggacctggggatgcggggcgggagagcgcggccggcgtgggagagctttatggggctgccccctcgatcgtctgcgaagtggggcgcggggcagtccctggaccggctctgctgtgctccgggcagtcgggggcttgcgggcgctccggggaggatgcgggcaccaccggctggacggtcgcagcctgcagggggccctggggacagcctgcctcatccccccggcgggctcgggccaggtggcagcgcctgggctcgccgcgcggaggcggcggcttctcgggctaggggcggcggcaggggcggccccgggatcacatgggcggaggcaggtccgggagccccgggcgggctctccccagagtcgggtagacggcagcgggagcggtggcgtctcctcgccttccctccctcccgggcctgggcgcccagccggacaggtgagcggcagccaggtgagcgcgcccacctgcgcctctccgcgcggcccgccctccccggcgccgggctcctctccgcgcccctgtcggcgcggaaccctggcctcgtccgcggcccagctccctggagcctcgcatcagcgggggcgcccccgcgagctgcgctctccccggccggagcgctcctcctccagcccccagcccgcagggtactttgccctcggagcgaaggaggctccagaactggtagagccgggccatcgggctgggcacctccccgcggcgcccgcagcgcggagtccactgaccggctcaaaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgccagggtctaagtagggtcgaacacagaagtgggaaggagaggggtgggccaggggctaatggtgtcactgtgtaaagtttttgacatactagctctataaatatatgaatatgg

SEQ ID NO: 33 - DNA, 1995 bp, Homo sapiens
atagaacgttttactggagaaccctgagagcagagggatgcgaggccaactgctggtagcagcctggtggggtgagggtctagggttgttttcttaagatccagaagtttttgctttagcttaaggatgtgtgcaattttccatgtggcttcataattcatccatgactttgaattttaaaatggagagaagttggcttcccaggaaatggtgcccctggccctgggcatcggcccacctggctgtctccaaggctctccttcccagtggctggtgcgggctccgggagctcagctgagtcccattggggtggcaacggaaagggagcaggggagtggggagtacggagatgcaggctggaaactggcctccaggaacaactgtgaggttgaaggcgagatttccatgttgggagcaaatttaagagccccagatggacacggcacacctggagacaggcactgctgcctgagtgtggcccagggagggctcggctccccaaagctccacggggcaggcccagacctcccagcagagtagggacaggctggttagcccagactcccgcctctgaggagaggaaccagctctccttgggaagagggcctctgccctggccggccctgactcccaggggacttcgaccccttgcttcatccctttatgtttctatctcctcagtgcaaaagggcagcgtgcttcctctctgctggttccgtgggatcggagccaggccccagtgtgcaatgtctttgcacacagataatagcgtgcgtgacagaggactctgtgcttagctgtcacctgctgccttcctgctgcccggtccccggcagaacccgcatgcctccctccctctctggaccctggcagtctctgcgccctggctccgagggaaggggcagggggcccgctggtcggcgagggctgggagcccccgacaccttggctccaagggaaggggcaggggtccgctggtcggcgagggccgggggccccctggtgtcgggcctgggaacagccgggctaggccctcctgggtgtgttggcccaaggctctggccctgactcctccccggctgcctcctcccaacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggac

SEQ ID NO: 34 - DNA, 1488 bp, Homo sapiens
aggcaagaattcggcacgaggaagcccctgagtagctgctccgggagctgtggtgccctctccttctccctttggctcctctcctgctgccttacagtgcccccagagccggctaggggcatggacttcacctcctggtcctctcttcctcaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctctagcagctccctcactggagaggcggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggcccccgacagtccagctcctttcggctcttgcaggaagccctggaggctgaggagagaggaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccatttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaataaaaaaaa

SEQ ID NO: 35 - DNA, 1530 bp, Homo sapiens
aggcaagaattcggcacgaggaagcccctgagtagctgctccgggagctgtggtgccctctccttctccctttggctcctctcctgctgccttacagtgcccccagagccggctaggggcatggacttcacctcctggtcctctcttcctcaggtatggcgttgacggtggatgtggccgggccagcgccctggggcttccgtatcacagggggcagggatttccacacgcccatcatggtgactaaggtggccgagcggggcaaagccaaggacgctgacctccggcctggagacataatcgtggccatcaacggggaaagcgcggagggcatgctgcatgccgaggcccagagcaagatccgccagagcccctcgcccctgcggctgcagctggaccggtctcaggctacgtctccagggcagaacaatggggacagctccttggaagtgctggcgactcgcttccagggctccgtgaggacatacactgagagtcagttctccttaaggtcctcctactccagcccaacctccctcagccgagggccggcagccccttctcaccaccaccctctagcagctccctcactggagaggcagccatcagccgcaggcggcctcggccgcgctggcgactcggcggtgctggtgctgccgccttccccgggccctcgttcctccaggcccagcatggactcggaagggggaagcctcctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcccaagctccacacttgtgagaagtgcagtaccagcatcgcgaaccaggctgtgcgcatccaggagggccggtaccgccaccccggctgctacacctgtgccgactgtgggctgaacctgaagatgcgcgggcacttctgggtgggtgacgagctgtactgtgagaagcatgcccgccagcgctactccgcacctgccaccctcagctctcgggcctgagcccgccatgccctcagcctgcctcactgctgggccagggtcatgcctatataagttggcatggcagggacaatggtgggcagttgctcttacatgagctaagtttggagacctgaggcccctttgtcctcgctgggtgggccaaggtctgggacctgtcttggactgtgggagactcaccctcaccttgccaggcctctcccctgcaggactggcattgcactagtctgaggtggccactgcctttgatcaacctttgtgtgcgagggtctaagtagggtcgaacacagaagtgggaaggagaggggtgggccaggggctaatggtgtcactgtgtaaagtttttgacatactagctctataaatatatgaatatggacaaaaaaaaaaaaaaaaa

SEQ ID NO: 36 - DNA, 1550 bp, Homo sapiens
gagcaactgaagaggcaggaggcagctgcgtgggctctcaactccccagggcaagagttgccaggcatggcgttgactgtggatgtggcaggaccagcaccttggggcttccgaattagcgggggcagagatttccacacacccatcattgtgaccaaggtcacagagcggggcaaggctgaagcagctgatctccggcctggcgacatcattgtggccatcaatggacagagtgcagagaacatgctacacgcggaggcccaaagcaagatccgacagagcgcctcacccctaagactgcagctggaccggtcccaaacagcctctcctgggcagaccaatggggagggctccttggaagtgctggcaaccagattccagggctccctgaggacacaccgtgacagccagtcttcccagaggtctgcctgcttcagcccagtctctctcagccccaggccttgcagccccttctccaccccaccccctaccagcccagttgccctttctaaagaggatatgattggctgtagtttccagagtctgacacactctccaggccttgctgctgctcaccacttgacctaccctggccaccccaccagccaacaggccggccacagcagcccaagcgactccgcagtgagggtgctgctccattccccaggacggccctccagccctaggttcagcagtttggatctggaggaagactcagaggtgttcaagatgctgcaggagaaccgccagggacgggccgccccaaggcagtccagctcttttcgactcttacaggaagccttggaggctgaggagagaggtggcacacctgcctttgtgcccagctcgctgagctcccaggcttccttgcccacctccagggccttggccactccacccaagctccacacctgtgagaaatgcagcgtcaacatctcgaaccaggcggtccgcatccaggaggggaggtaccgacaccctggctgctacacttgcgcagactgtgggctgaacctgaagatgcgcggccacttctgggtgggcaatgagttgtactgcgagaagcatgcccgccagcgctactctatgcctggaactctcaactctcgagcctgagcctcaaggtgctcggcctgtctgcactctcagactctgcagacatgattatactgagagcaagcagggaaggggtgatagcaggtgatagatgatcttacatgaactaaggttggggagtcccctttgtccttgctgggtgaggccaagggttgggactaatgtcaggttgctagtgctaaggacagttccactctctctggccttcctcctgcaggccaggttctgtattacggtctacagtggctgccatgtttgacacgaaagcgtatggggttgggcatggatagaagcatctagaagggaatggtgggcctgaggtaaatgatattcatggtgtgaagtttctaacatatgaactctatatacacgtggataaaattaagtagtgtattttcaaaaaaaaaaaaaaaaaaaaaaaaaaaaaaaaaaaa

SEQ ID NO: 37 - DNA, 1540 bp, Homo sapiens
gagcaactgaagaggcaggaggcagctgcgtgggctctcaactccccagggcaagagttgccaggcatggcgttgactgtggatgtggcaggaccagcaccttggggcttccgaattagcgggggcagagatttccacacacccatcattgtgaccaaggtcacagagcggggcaaggctgaagcagctgatctccggcctggcgacatcattgtggccatcaatggacagagtgcagagaacatgctacacgcggaggcccaaagcaagatccgacagagcgcctcacccctaagactgcagctggaccggtcccaaacagcctctcctgggcagaccaatggggagggctccttggaagtgctggcaaccagattccagggctccctgaggacacaccgtgacagccagtcttcccagaggtctgcctgcttcagcccagtctctctcagccccaggccttgcagccccttctccaccccaccccctaccagcccagttgccctttctaaagaggatatgattggctgtagtttccagagtctgacacactctccaggccttgctgctgctcaccacttgacctaccctggccaccccaccagccaacaggccggccacagcagcccaagcgactccgcagtgagggtgctgctccattccccaggacggccctccagccctaggttcagcagtttggatctggaggaagactcagaggtgttcaagatgctgcaggagaaccgccagggacgggccgccccaaggcagtccagctcttttcgactcttacaggaagccttggaggctgaggagagaggtggcacacctgcctttgtgcccagctcgctgagctcccaggcttccttgcccacctccagggccttggccactccacccaagctccacacctgtgagaaatgcagcgtcaacatctcgaaccaggcggtccgcatccaggaggggaggtaccgacaccctggctgctacacttgcgcagactgtgggctgaacctgaagatgcgcggccacttctgggtgggcaatgagttgtactgcgagaagcatgcccgccagcgctactctatgcctggaactctcaactctcgagcctgagcctcaaggtgctcggcctgtctgcactctcagactctgcagacatgattatactgagagcaagcagggaaggggtgatagcaggtgatagatgatcttacatgaactaaggttggggagtcccctttgtccttgctgggtgaggccaagggttgggactaatgtcaggttgctagtgctaaggacagttccactctctctggccttcctcctgcaggccaggttctgtattacggtctacagtggctgccatgtttgacacgaaagcgtatggggttgggcatggatagaagcatctagaagggaatggtgggcctgaggtaaatgatattcatggtgtgaagtttctaacatatgaactctatatacacgtggataaaattaagtagtgtattttcaaaaaaaaaaaaaaaaaaaaaaaaaa

SEQ ID NO: 38 - DNA, 1223 bp, Homo sapiens
acggcagcgggagcggtggcgtctcctcgccttccctccctcccgggcctgggcgcccagccggacaggtatggcgttgacggtggatgtggccgggccagcgccctggggcttccgtatcacagggggcagggatttccacacacccatcatggtgactaaggcagccattttgggtgattactagaacaagagccccggagttcttggcagtctcctcttgacctgggcccaggctgggaacgttcacaggagggctgtgcaggcagccacctgcaaggtggccgagcggggcaaagccaaggacgctgacctccggcctggagacataatcgtggccatcaacggggaaagcgcggagggcatgctgcatgctgaggcccagagcaagatccgccagagcccctcgcccctgcggctgcagctggaccggtctcaggctacgtctccagggcagaccaatggggacagctccttggaagtgctggcgactcgcttccagggctccgtgaggacatacactgagagtcagtcctccttaaggtcctcctactccagcccaacctccctcagcccgagggccggcagccccttctcaccaccaccctccagcagccccctcactggagaggtggccatcagccgcagcttccagagtctggcatgttccccgggcctccccgctgctgaccgcctgtcctactcaggccgccctggaagccgacaggccggcctcggccgcgctggcgactcggcggtgctggtgctgccgccttccccgggccctcgttcctccaggcccagcatggactcggaagggggaagccttctcctggacgaggactcggaagtcttcaagatgctgcaggaaaatcgcgagggacgggcggccccccgacagtccagctcctttcggctcttgcaggaagccctggaggctgaggagagaggtggcacgccagccttcttgcccagctcactgagcccccagtcctccctgcccgcctccagggccctggccacccctcaccttgccaggcctctcccctgcaggactggcattgcactagtctgaggtggccactgcctttgatcaacctttgtgtgccagggtctaagtagggtcgaacacagaagtgggaaggagaggggtgggccaggggctaatggtgtcactgtgtaaagtttttgacattctacccccataaatatatgaatatgga

SEQ ID NO: 39 - DNA, 804 bp, Homo sapiens
atggcagtggcttaccttgtcaggaacgtgcggaggggctgcccctcccgttccttcaagcatcactcagtcctggcctggttctaggcttggggctgtggctgccacaggaagggggatacccccatactttgggtgttgggtcagatcctgcccccgcgccccacagggctcaggttcctgtgagctgatcctgggctaggtcgtatctgtgtggggaggggagaggggtgcaccatggcctggaggggagtgtgtgttggggtgtgtgtgtgtggaggttcttttgagtcacacacaaagcattgtgctgagacactgcattcctgctggctgggcttcctgtttccaaatgcattcctgccccagagtcaccacagagactgtttggaatcctgccccacattctccaaattcagggcgtgatctggcaaaggctccctcccccaccctcttactttggagggactctgggctccctcctaaccagggagggctggcagggctctggcacggcagtgagtgtgatgccacacatcttcttgggcttgtataaggcagttcctgcaggcccaccactctggttctctctgcctgtgggaagcagaatgggtactatctaggaggactgggctgggagggggcaacccagaggacccatggctagcagggaagggcctgggcttgaacatgactctgttgagacatggccagcaattccggcacttggctgtcattcagccggcctgcacagggcttggccgtgggcctcagtttccccattttataaagttttaaaatctg

SEQ ID NO: 40 - DNA, 1488 bp, Homo sapiens; identical to SEQ ID NO: 34

SEQ ID NO: 41 - protein, 352 aa, Homo sapiens; identical to SEQ ID NO: 2

SEQ ID NO: 42 - protein, 278 aa, Homo sapiens; identical to SEQ ID NO: 5

SEQ ID NO: 43 - protein, 366 aa, Homo sapiens; identical to SEQ ID NO: 6

SEQ ID NO: 44 - protein, 289 aa, Homo sapiens; identical to SEQ ID NO: 7

SEQ ID NO: 45 - protein, 325 aa, Homo sapiens; identical to SEQ ID NO: 3

SEQ ID NO: 46 - protein, 349 aa, Homo sapiens; identical to SEQ ID NO: 8

SEQ ID NO: 47 - protein, 349 aa, Homo sapiens; identical to SEQ ID NO: 8

SEQ ID NO: 48 - protein, 236 aa, Homo sapiens; identical to SEQ ID NO: 9

SEQ ID NO: 49 - protein, 352 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQTNGDSSLEVLATRFQGSVRTYTESQSSLRSSYSSPTSLSPRAGSPFSPPPSSSSLTGEAAISRSFQSLACSPGLPAADRLSYSGRPGSRQAGLGRAGDSAVLVLPPSPGPRSSRPSMDSEGGSLLLDEDSEVFKMLQENREGRAAPRQSSSFRLLQEALEAEERGGTPAFLPSSLSPQSSLPASRALATPPKLHTSEKSSTSIANQAVRIQEGRYRHPGCYTCADCGLNLKMRGHFWVGDELYCEKHARQRYSAPATLSSRA

SEQ ID NO: 50 - protein, 52 aa, Homo sapiens
CEKCSTSIANQAVRIQEGRYRHPGCYTCADCGLNLKMRGHFWVGDELYCEKH

SEQ ID NO: 51 - protein, 52 aa, Homo sapiens
SEKSSTSIANQAVRIQEGRYRHPGCYTCADCGLNLKMRGHFWVGDELYCEKH

SEQ ID NO: 52 - protein, 52 aa, Homo sapiens
CEKCSTSIANQAVRIQEGRYRHPGSYTSADCGLNLKMRGHFWVGDELYCEKH

SEQ ID NO: 53 - protein, 52 aa, Homo sapiens
SEKSSTSIANQAVRIQEGRYRHPGSYTSADCGLNLKMRGHFWVGDELYCEKH